(19) United States
(12) Patent Application Publication
(10) Pub. No.: US 2026/0165615 A1
(43) Pub. Date: Jun. 18, 2026

(54) SYSTEMS, METHODS, AND DEVICES FOR DIAGNOSTICS BASED ON LIVE UN-SCATTERING COMPUTATIONAL IMAGING

(71) Applicant: oselva, Inc., San Francisco, CA (US)

(72) Inventors: Hervé Dominique MARIE-NELLY, San Francisco, CA (US); Shengjiang TU, Foster City, CA (US)

(73) Assignee: oselva, Inc., San Francisco, CA (US)

(21) Appl. No.: 19/416,980

(22) Filed: Dec. 11, 2025

Related U.S. Application Data
(60) Provisional application No. 63/733,334, filed on Dec. 12, 2024.

(51) Int. Cl. A61B 5/1455; A61B 5/00; A61B 5/145; G06T 7/00; G06V 10/143; G06V 10/25; G06V 10/774; G06V 20/69

(52) U.S. Cl. CPC A61B 5/1455; A61B 5/14546; A61B 5/7267; G06T 7/0012; G06T 12/10; G06V 10/143; G06V 10/25; G06V 10/774; G06V 20/693; G06V 20/695; A61B 2562/0238; G06T 2207/10048; G06T 2207/10064; G06T 2207/10068; G06T 2207/10088; G06T 2207/10132; G06T 2207/20081; G06T 2207/30041; G06T 2207/30048; G06T 2207/30088; G06T 2211/441; G06T 2211/452; G06V 2201/03

(57) ABSTRACT

Methods for identifying micron-scale features below a surface of a tissue include emitting from one or more emitters at the surface of the tissue electromagnetic signals capable of penetrating tissue of different densities; obtaining from a sensor positioned at the surface of the tissue electromagnetic signals reflected from or transmitted through tissues of different densities as one or more obtained images; inputting the one or more obtained images into a machine learning model, wherein the machine learning model is trained to: identify scattering of certain electromagnetic signals comprising each of the one or more obtained images; correct for the scattering to create one or more images tissues at one or more subsurface depths; and identify micron-scale features of interest within the one or more images of tissues at the one or more subsurface depths.

### CROSS-REFERENCE TO RELATED APPLICATIONS

This application claims the benefit of U.S. Provisional Application No. 63/733,334, filed Dec. 12, 2024, the entire contents of which is incorporated herein by reference.

### FIELD

This disclosure relates generally to medical image processing and more specifically to machine-learning based medical image processing.

### BACKGROUND

Deep-tissue (e.g., 1-2-centimeter depth) live imaging of in vivo human samples is a particularly challenging task due to the absorption, reflection and scattering properties of the skin and subcutaneous tissues, such as bone and blood vessels. Current methods are limited in depth and/or resolution and often require specialized equipment and human operators. Existing methods include optical coherence tomography (OCT), ultrasound, magnetic resonance imaging (MRI), and histopathology imaging. OCT provides high resolution images but is limited to a depth of between 5 millimeters to 1 centimeter. Additionally, OCT cannot be accomplished through a portable or wearable device. Ultrasound offers high-depth imaging, but provides low resolution images, requires a skilled technician, and demands very close contact and gel to function. MRI offers high-resolution, high depth, non-invasive imaging, but is expensive and requires access to an MRI-equipped clinical facility and a skilled technician for image acquisition. Moreover, MRI does not support micro-level resolution for deeper sample phenotyping. Finally, histopathology imaging provides high lateral resolution and is compatible with other molecular imaging techniques, but it is not suitable for live, in vivo sampling and requires chemical- or cryo-fixation of the sample. Further, histopathology imaging is expensive and offers low axial resolution.

### SUMMARY

Disclosed herein are systems, devices, and methods that employ machine-learning based medical image processing techniques. The disclosed systems, devices, and methods may be used for identifying features of interest below the surface of a tissue at one or more subsurface depths. The systems, devices, and methods disclosed herein may emit electromagnetic signals capable of penetrating tissue and other structures of different densities. The electromagnetic signals may be emitted from a surface of a tissue and may include Near Infrared (NIR) and/or Short-Wave Infrared (SWIR) signals. The signals may be reflected from or transmitted through the tissues and obtained as such, or as images, using one or more sensors. Some of the signals may scatter as they propagate within the tissue, degrading image quality. The systems disclosed herein may create high-resolution (e.g., 1 micron resolution) images from the obtained signals or images. For instance, machine learning models may be trained to reconstruct high-resolution images at one or more subsurface depths that correct for electromagnetic signal scattering. The machine learning models may be trained to identify features of interest based on the signals detected by the sensor(s) and/or the reconstructed images. Micron-scale imaging (e.g., as high as single micrometer resolution) enables the systems disclosed herein to distinguish between normal and inflamed nerves, identify microvascular damages, swollen lymphatic vessels, protein accumulations, lipid accumulations, and other structures and microstructures, enabling early detection of various medical conditions and disease states. The systems and devices may include portable, low-cost, and/or non-invasive imaging techniques and may be utilized by consumers or medical specialists.

According to an aspect, an exemplary method for identifying micron-scale features below a surface of a live human tissue comprises: emitting from one or more emitters at the surface of the live human tissue electromagnetic signals capable of penetrating human tissue of different densities; obtaining from a sensor positioned at the surface of the live human tissue electromagnetic signals reflected from or transmitted through live human tissues of different densities as one or more obtained images; inputting the one or more obtained images into a machine learning model, wherein the machine learning model is trained to: identify scattering of certain electromagnetic signals comprising each of the one or more obtained images; correct for the scattering to create one or more images of live human tissues at one or more subsurface tissue depths; and identify micron-scale features of interest within the one or more images of live human tissues at the one or more subsurface tissue depths.

Optionally, the emitted electromagnetic signals are comprised of near infrared or short-wave infrared light. Optionally, the method comprises modifying a scattering of the electromagnetic signals obtainable by the sensor using a microwave phase array positioned at the surface of the live human tissue. Optionally, the method comprises adjusting at least one of an illumination strength and an illumination parameter of the one or more emitters to modify a scattering of the electromagnetic signals obtainable by the sensor. Optionally, the one or more emitters comprise one or more coherent light sources. Optionally, the one or more emitters comprise an optical phase array. Optionally, the sensor comprises a camera configured to capture the near infrared spectrum and the short-wave infrared spectrum. Optionally, the camera is configured to capture at least part of the visible spectrum. Optionally, the sensor comprises an optical phase array configured to capture the near infrared spectrum and the short-wave infrared spectrum. Optionally, the micron scale feature of interest is any of: a protein accumulation below the surface of the live human tissue, a lipid accumulation below the surface of the live human tissue, and a spectral response of a tissue to the electromagnetic signals.

Optionally, the machine learning model has been trained by: associating training images of known protein accumulations with subdermal training images obtained from a sensor positioned at a surface of a live human tissue. Optionally, the machine learning model has been trained by: training the machine learning model to reconstruct training images of known protein accumulations. Optionally, the training images of known protein accumulations comprise any one or more of: a fluorescent image; a magnetic resonance imaging (MRI) image; an ultrasound image; and a histopathology image. Optionally, the machine learning model has been trained to reconstruct from the one or more obtained or created images a three-dimensional model of an anatomy below the surface of the tissue. Optionally, the machine learning model has been trained to deconvolve a scatter present in the electromagnetic signals received by the one or more image sensors.

Optionally, the one or more image sensors are included on at least one of: a wearable device and an endoscope. Optionally, the method comprises predicting a disease state based on the micron-scale feature of interest. Optionally, the disease state comprises any of cardiac amyloidosis, carpal tunnel syndrome, light chain amyloidosis, a peripheral neuropathy, or any combination thereof. Optionally, the protein accumulations below the surface of the tissue are located below the surface of any of: a wrist, an eye, a finger, and a nose. Optionally, the micron-scale feature of interest is at least one micrometer in size. Optionally, the surface of the live human tissue is the surface of a skin, a heart, or an eye. Optionally, the one or more obtained images comprise a plurality of images, and wherein the machine learning model is trained to correct for the scattering to create a plurality of images of live human tissues at a plurality of subsurface tissue depths.

According to an aspect, an exemplary system for identifying micron-scale features below a surface of a live human tissue comprises: one or more processors, a memory, and one or more programs, wherein the one or more programs are stored in the memory and configured to be executed by the one or more processors, the one or more programs including instructions for: emitting from one or more emitters at the surface of the live human tissue electromagnetic signals capable of penetrating human tissue of different densities; obtaining from a sensor positioned at the surface of the live human tissue electromagnetic signals reflected from or transmitted through live human tissues of different densities as one or more obtained images; inputting the one or more obtained images into a machine learning model, wherein the machine learning model is trained to: identify scattering of certain electromagnetic signals comprising each of the one or more obtained images; correct for the scattering to create one or more images of live human tissues at one or more subsurface tissue depths; and identify micron-scale features of interest within the one or more images of live human tissues at the one or more subsurface tissue depths.

According to an aspect, an exemplary non-transitory computer-readable storage medium stores one or more programs for identifying micron-scale features below a surface of a live human tissue, the one or more programs comprising instructions, which when executed by one or more processors of an electronic device, cause the electronic device to: emit from one or more emitters at the surface of the live human tissue electromagnetic signals capable of penetrating human tissue of different densities; obtain from a sensor positioned at the surface of the live human tissue electromagnetic signals reflected from or transmitted through live human tissues of different densities as one or more obtained images; input the one or more obtained images into a machine learning model, wherein the machine learning model is trained to: identify scattering of certain electromagnetic signals comprising each of the one or more obtained images; correct for the scattering to create one or more images of live human tissues at one or more subsurface tissue depths; and identify micron-scale features of interest within the one or more images of live human tissues at the one or more subsurface tissue depths.

According to an aspect, an exemplary device for identifying micron-scale features below a surface of a live human tissue comprises: one or more emitters at the surface of the live human tissue configured to emit electromagnetic signals capable of penetrating human tissue of different densities; at least one sensor positioned at the surface of the live human tissue configured to detect electromagnetic signals reflected from or transmitted through live human tissues of different densities as one or more obtained images; one or more processors; and a memory storing computer instructions that when executed by the one or more processors, cause the device to: input the one or more obtained images into a machine learning model, wherein the machine learning model is trained to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

According to an aspect, an exemplary method of training a machine learning model to identify micron-scale features from a scan of live human tissues comprises: obtaining one or more images based on electromagnetic signals reflected from or transmitted through live human tissues of different densities; inputting the one or more obtained images into the machine learning model; and training the machine learning model to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

Optionally, the micron scale feature of interest is any of: a protein accumulation, a lipid accumulation, and a spectral response of a tissue to the electromagnetic signals. Optionally, training the machine learning model comprises: associating training images of known protein accumulations or lipid accumulations with subdermal training images obtained from a sensor positioned at the surface of a live human tissue. Optionally, training the machine learning model comprises: training the machine learning model to reconstruct training images of known protein accumulations or lipid accumulations. Optionally, the training images of known protein accumulations or lipid accumulations comprise any one or more of: a fluorescent image; a magnetic resonance imaging (MRI) image; an ultrasound image; and a histopathology image. Optionally, the training images comprise a training image of a first type depicting a region of a live human tissue and a training image of a second type depicting the same region of the live human tissue. Optionally, at least a subset of the training images are labeled to indicate any of: a disease state, a tissue classification, a biomarker, a segmentation mask of a disease feature, a protein accumulation, a lipid accumulation, and a physiological parameter. Optionally, training the machine learning model comprises: training the machine learning model to reconstruct from the obtained or created images a three-dimensional model of an anatomy below the surface of the tissue Optionally, training the machine learning model comprises: training the machine learning model to deconvolve a scatter present in the electromagnetic signals.

Optionally, obtaining the plurality of images comprises: emitting from one or more emitters at the surface of the live human tissue electromagnetic signals capable of penetrating human tissue of different densities; obtaining from a sensor positioned at the surface of the live human tissue electromagnetic signals reflected from or transmitted through live human tissues of different densities as the one or more obtained images Optionally, obtaining the one or more images comprises adjusting at least one of an illumination strength and an illumination parameter of the one or more emitters to modify a scattering of the electromagnetic signals obtainable by the sensor. Optionally, the illumination parameter comprises any one or more of: an illumination angle, a polarization, a frequency, and a target depth. Optionally, obtaining the one or more images comprises modifying a scattering of the electromagnetic signals obtainable by the sensor using a microwave phase array positioned at the surface of the live human tissue. Optionally, the one or more emitters comprise one or more coherent light sources. Optionally, the one or more emitters comprises an optical phase array. Optionally, the sensor comprises a camera configured to capture the near infrared spectrum and the short-wave infrared spectrum. Optionally, the camera is configured to capture at least part of the visible spectrum. Optionally, the sensor comprises an optical phase array configured to capture the near infrared spectrum and the short-wave infrared spectrum. Optionally, the electromagnetic signals are comprised of near infrared or short-wave infrared light. Optionally, the micron scale feature of interest is any of: a protein accumulation below the surface of the live human tissue, a lipid accumulation below the surface of the live human tissue, and a spectral response of a tissue to the electromagnetic signals. Optionally, the one or more obtained images comprise a plurality of images, and wherein the machine learning model is trained to correct for the scattering to create a plurality of images of live human tissues at a plurality of subsurface tissue depths.

According to an aspect, an exemplary system for training a machine learning model to identify micron-scale features from a scan of live human tissues, the system comprising: one or more processors, a memory, and one or more programs, wherein the one or more programs are stored in the memory and configured to be executed by the one or more processors, the one or more programs including instructions for: obtaining one or more images based on electromagnetic signals reflected from or transmitted through live human tissues of different densities; inputting the one or more obtained images into the machine learning model; and training the machine learning model to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

According to an aspect, an exemplary non-transitory computer-readable storage medium stores one or more programs for identifying micron-scale features below a surface of a live human tissue, the one or more programs comprising instructions, which when executed by one or more processors of an electronic device, cause the electronic device to: obtain one or more images based on electromagnetic signals reflected from or transmitted through live human tissues of different densities; input the one or more obtained images into the machine learning model; and train the machine learning model to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

In some embodiments, any one or more of the characteristics of any one or more of the systems, methods, and/or computer-readable storage mediums recited above may be combined, in whole or in part, with one another and/or with any other features or characteristics described elsewhere herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

A better understanding of the features and advantages of the present disclosure will be obtained by reference to the following detailed description that sets forth illustrative embodiments, in which the principles of the disclosure are utilized, and the accompanying drawings of which:

FIG. 1A illustrates aspects a system for identifying features of interest beneath a surface of a live human tissue according to some examples.

FIG. 1B illustrates additional aspects that may be included in the system of FIG. 1A according to some examples.

FIG. 2A illustrates a device for identifying features of interest beneath a surface of a tissue according to some examples.

FIG. 2B illustrates aspects of another device illustrates a device for identifying features of interest beneath a surface of a tissue according to some examples.

FIG. 3 illustrates a method for training a machine learning model according to some examples.

FIG. 4 illustrates a method for training a machine learning model to identify micron-scale features of interest within the one or more images of live human tissues at one or more subsurface tissue depths according to some examples.

FIG. 5 illustrates a method for training a machine learning model to associate imaging settings of at least one of an emitter and an optical phase clearer with different amounts of scattering of electromagnetic signals at different tissue regions according to some examples.

FIG. 6 illustrates effects of the imaging settings of the emitter and optical phase clearer on signal scattering according to some examples.

FIG. 7 illustrates an exemplary method for identifying micron-scale features of interest within one or more images of live human tissues at one or more subsurface tissue depths according to some examples.

FIG. 8 illustrates a latent diffusion machine learning model architecture according to some examples.

FIG. 9 illustrates a computing device according to some examples.

### DETAILED DESCRIPTION

Disclosed herein are systems, devices, and methods for identifying features of interest below the surface of a tissue using machine learning techniques. The systems, devices, and methods disclosed herein may enable early detection of features associated with different disease states. Thus, the disclosed systems, devices, and methods may enable early diagnosis, treatment and lower severity therapeutic interventions. The disclosed systems, methods, and devices may obtain as images, using one or more sensors, electromagnetic signals emitted into tissues from the surface of the tissue. The obtained images may be used to train one or more machine learning models. For instance, the one or more machine learning models may be trained to associate the obtained images with ground-truth images (e.g., fluorescent images, complex refractive index maps, MRI images, ultrasound images) of the same regions of the tissue. The one or more machine learning models may be trained to predict features below the surface of the tissue based on the obtained images and the one or more ground truth images. The one or more machine learning models may be trained to identify features of interest, such as protein accumulations, lipid accumulations, lymphatic structures, spectral responses of different tissues to the electromagnetic signals, and other structures, microstructures, etc. beneath the surface of a tissue based on the obtained images. The one or more trained machine learning models may predict disease state(s) based on the features of interest. The one or more machine learning models may be trained to learn time variant features, such as disease progression, morphological changes, etc. based on time series of obtained images. The one or more machine learning models be trained to create one or more high-resolution (e.g., 1 micrometer resolution images) based on the obtained images. The one or more trained machine learning models may be trained in a supervised or self-supervised fashion to create representations (e.g., embedding vectors) of the tissue. These embeddings may be used for image, signal generations or classification tasks.

The one or more machine learning models may be trained to determine optimized imaging settings to reduce (e.g., minimize) scattering of electromagnetic signals as they propagate within the tissue. The one or more machine learning models disclosed herein may be trained as specialized models optimized for a given patient population, particular organs or region within the body, a particular disease state, a particular tissue structure or microstructure, a particular spectral response associated with a certain type of protein accumulation, particular imaging devices, etc. Imaging settings may be determined by the one or more machine learning models depending on a task (e.g., the region of the body being imaged, a particular disease state being targeted, etc.) and the determined settings may be applied by the systems disclosed herein during image acquisition.

In some examples, the emitter source positions or other optical parameters of the device may have some amount of (intrinsic) error (e.g., due to manufacturing defects) and non-static error (e.g. temperature changes caused by electronics or environmental conditions) that may be estimated prior to imaging. The devices disclosed herein may be adjusted to account for the intrinsic deviations. The system may also detect if certain conditions are satisfied for the imaging, including safety for the target (e.g., total power sent kept below a certain limit) and device geometry/orientation. The system may be configured to alert a user regarding whether the conditions are met or not (e.g., binary output). The system may self-adjust the imaging settings based on determination of whether the certain conditions including safety for the target and/or device orientation/geometry are satisfied.

Once trained, the one or more machine learning models may be used for various inference tasks based on images obtained by emitting electromagnetic signals into a tissue from the surface of the tissue. For instance, a sensor may be positioned at the surface of a tissue and electromagnetic signals reflected from or transmitted through live human tissues of different densities may be obtained using the sensor as one or more obtained images. The obtained images may be input into one or more trained machine learning models. In some examples, the electromagnetic signals may be obtained as such using the sensor, and one or more images may be constructed from such electromagnetic signals after input into one or more trained machine learning models. The one or more trained machine learning models may create high-resolution images (e.g., 1 micrometer resolution images) based on the obtained images or obtained signals. The one or more trained machine learning models may identify features of interest, including but not limited to protein accumulations, lipid accumulations, inflamed nerves, lymphatic structures, spectral responses of different tissues to the electromagnetic signals, and other structures, microstructures, etc. beneath the surface of a tissue, based on the obtained images or the obtained signals. The one or more trained machine learning models may predict disease state(s) based on the identified features of interest. The one or more trained machine learning models may model disease progressions based on the identified features of interest. The one or more trained machine learning models may determine optimized imaging settings to reduce (e.g., minimize) scattering of electromagnetic signals as they propagate within the tissue.

An exemplary system may include one or more emitters positioned at the surface of the live human tissue. The emitters may be configured to emit electromagnetic signals capable of penetrating tissue of different densities. At least one sensor may be positioned at the surface of the tissue and may be configured to detect electromagnetic signals reflected from and/or transmitted through live human tissues of different densities as such, or as one or more obtained images. The exemplary system may input the detected electromagnetic signals or the one or more obtained images into one or more machine learning models. The one or more machine learning models may be trained to identify scattering of certain electromagnetic signals obtained as such, or comprising each of the one or more obtained images and correct for such scattering to create one or more images of tissues at one or more subsurface tissue depths. The one or more machine learning models may be trained to identify micron-scale features (e.g., objects separated by as few as 1 micrometer) of interest within the one or more images of live human tissues at the one or more subsurface tissue depths. Signal scattering may be addressed during imaging via specific configuration of imaging settings, including illumination strength, illumination parameters (e.g., angle, polarization, etc.), and emissions from an optical phase clearer. Additionally, or alternatively, signal scattering can be addressed computationally after obtaining electromagnetic signals reflected from or transmitted through the sample.

The systems, devices, and methods disclosed herein provide numerous technological advantages. The disclosed systems, devices, and methods unlock high resolution (e.g., 1 micrometer resolution), high depth (e.g., at least 1 to 2 centimeters), low phototoxicity, and live imaging of tissues. The disclosed systems, devices, and methods enable the identification of micron-scale features of interest (e.g., objects separated by as few as 1 micrometer) beneath the surface of tissues using portable and non-invasive imaging techniques. Moreover, the systems, devices, and methods disclosed herein enable both morphological imaging of samples and the spectral characterization of molecular entities.

Exemplary systems can be integrated in wearable non-invasive diagnostic devices to characterize the nervous, cardiovascular, and lymphatic system at high resolution. Exemplary systems can also be integrated into various existing medical imaging devices, such as endoscopes, flexscopes, and other medical imaging devices typically deployed in clinical settings. The systems disclosed herein may be used to image live human tissues and/or other live mammalian tissues. Live imaging in human tissues and other mammalian tissues enables early detection and intervention for various disease states that may otherwise go undetected. The portable and low-cost nature of various exemplary systems disclosed herein additionally expand access to the technical benefits provided by the disclosed systems and methods. As an example, the systems, devices, and methods disclosed herein enable pre-symptomatic detection of various pathologies, such as ATTR, and early symptomatic confirmation of disease onset. In turn, such early diagnosis will allow for early treatment and lower severity therapeutic interventions.

In the following description of the various embodiments, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The structure for a variety of these systems will appear from the description below. In addition, the present invention is not described with reference to any particular programming language. It will be appreciated that a variety of programming languages may be used to implement the teachings of the present disclosure as described herein.

### Exemplary System for Identifying Features Below the Surface of a Tissue

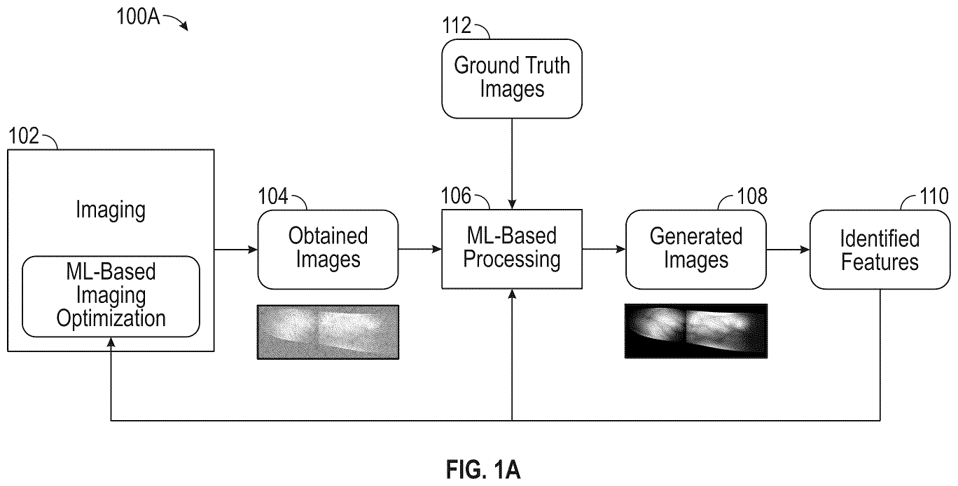

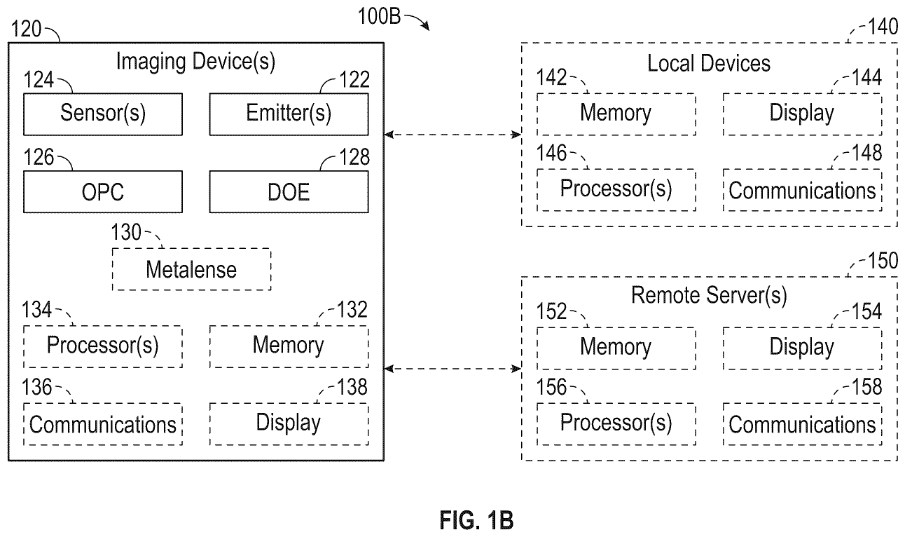

FIG. 1A illustrates an exemplary system 100A for identifying features below a surface of a tissue. These features can be less than 1000 microns, less than 100 microns, and/or as small as one micron in size. System 100A includes an imaging stage 102 and a machine-learning (ML) based processing stage 106. During imaging stage 102, electromagnetic signals capable of penetrating tissue and other biological structures of different densities may be emitted from one or more emitters at the surface of the tissue. The emitted electromagnetic signals may be comprised of near infrared or short-wave infrared light. System 100A may obtain from a sensor positioned at the surface of the tissue, electromagnetic signals reflected from or transmitted through tissues and other anatomical structures and microstructures of different densities as one or more obtained images 104. The tissue may be a live mammalian tissue such as a live human tissue. The surface of the tissue may be, for instance, the surface of a skin, a heart, or an eye. System 100A may enable imaging at as high as 1 micrometer resolution, as high as 10 micrometer resolution, and/or as high as 1000 micrometer resolution. System 100A may enable identification of objects separated by as few as 1 micron, as few as 10 microns, as few as 100 microns, and/or as few as 1000 microns.

The emitted electromagnetic signals may be emitted at a wavelength between 700 nanometers (nm) and 3000 nanometers (nm). The emitted electromagnetic signals may be transmitted as a swept signal comprising a plurality of different wavelengths. The emitted electromagnetic signals may be at least 700 nanometers (nm), at least 800 nanometers (nm), at least 900 nanometers (nm), at least 1000 nanometers (nm), at least 1100 nanometers (nm), at least 1200 nanometers (nm), at least 1300 nanometers (nm), at least 1400 nanometers (nm), at least 1500 nanometers (nm), at least 1600 nanometers (nm), at least 1700 nanometers (nm), at least 1800 nanometers (nm), at least 1900 nanometers (nm), at least 2000 nanometers (nm), at least 2100 nanometers (nm), at least 2200 nanometers (nm), at least 2300 nanometers (nm), at least 2400 nanometers (nm), at least 2500 nanometers (nm), at least 2600 nanometers (nm), at least 2700 nanometers (nm), at least 2800 nanometers (nm), at least 2900 nanometers (nm), and/or at least 3000 nanometers (nm).

The emitted electromagnetic signals may be at most 700 nanometers (nm), at most 800 nanometers (nm), at most 900 nanometers (nm), at most 1000 nanometers (nm), at most 1100 nanometers (nm), at most 1200 nanometers (nm), at most 1300 nanometers (nm), at most 1400 nanometers (nm), at most 1500 nanometers (nm), at most 1600 nanometers (nm), at most 1700 nanometers (nm), at most 1800 nanometers (nm), at most 1900 nanometers (nm), at most 2000 nanometers (nm), at most 2100 nanometers (nm), at most 2200 nanometers (nm), at most 2300 nanometers (nm), at most 2400 nanometers (nm), at most 2500 nanometers (nm), at most 2600 nanometers (nm), at most 2700 nanometers (nm), at most 2800 nanometers (nm), at most 2900 nanometers (nm), and/or at most 3000 nanometers (nm).

The one or more emitters used during the imaging stage 102 may include one or more coherent light sources and/or an optical phase array. The sensor may include a camera configured to capture the near infrared spectrum and the short-wave infrared spectrum. The camera may be configured to capture at least part of the visible spectrum. The sensor may include an optical phase array configured to capture the near infrared spectrum and the short-wave infrared spectrum. The emitter(s) and/or sensor(s) may be included on a wearable device such as a ring, wristband, watch, or eyeglasses, a medical device such as an endoscope, a flex-scope, or any other imaging device. The one or more emitters and/or the optical phase array may be programmable such that imaging settings, including illumination strength, illumination parameters (e.g., angle, polarization, frequency, target depth, polarization setup, focus settings, etc.), and microwave emissions from the optical phase clearer can be adjusted, optionally in real time during imaging, to modify (e.g., minimize) scattering of the electromagnetic signals.

During the ML-based processing stage 106, system 100A may input the obtained images 104 into one or more machine learning models. The one or more machine learning models may include one or more latent diffusion models, one or more diffusion transformer models, one or more transformer models, one or more diffusion models, one or more convolutional neural networks, etc. The one or more machine learning models may include a plurality of machine learning models merged in an end-to-end manner. The one or more machine learning models may include a machine learning model with a plurality of task specific heads. During a training phase of ML-based processing stage 106, the one or more machine learning models may be trained to associate the obtained images 104 with ground-truth images 112 (e.g., fluorescent images, MRI images, ultrasound images) of the same region(s) of the tissue. Training the one or more machine learning models may include training the machine learning model to reconstruct the ground truth images 112 of known structures and/or microstructures, including any one or more of protein accumulations, lipid accumulations, lymphatic structures, nerves, etc. The ground truth images 112 and the obtained images 104 may depict the same region of tissue.

The one or more machine learning models may be trained to predict structural features below the surface of the tissue based on the obtained images 104 and the one or more ground truth images 112. The one or more machine learning models may be trained to identify features of interest 110, such as structures and/or microstructures including any one or more of protein accumulations, lipid accumulations, lymphatic structures, nervous system structures, spectral responses of different tissues to the electromagnetic signals, etc. beneath the surface of a tissue based on the obtained images 104. The one or more trained machine learning models may predict disease state(s) based on the features of interest 110. The one or more machine learning models may additionally, or alternatively, be trained to learn time variant features, such as disease progression, morphological changes, etc. based on time series of obtained images 104. The one or more machine learning models be trained to may create one or more high-resolution (e.g., 1 micrometer resolution images) based on the obtained images 104.

In some examples, the one or more machine learning models may be trained to identify scattering of certain electromagnetic signals comprising each of the one or more obtained images 104 and correct for the scattering to create one or more generated images 108 of live human tissues at one or more subsurface tissue depths. The one or more machine learning models may be trained to identify features of interest 110 (e.g., micron-scale features of interest) within the one or more generated images 108 of live human tissues at the one or more subsurface tissue depths, such as structures and microstructures including any one or more of protein accumulations, lipid accumulations, nerves, blood vessels, glands, lymph nodes and other structures of the lymphatic system, etc. The features of interest 110 may be located below the surface of a wrist, an eye, a finger, a nose, a blood vessel, etc. The machine learning model may be trained to reconstruct, from the obtained images 104 or generated images 108, a three-dimensional model of an anatomy below the surface of the tissue.

The one or more machine learning models may be trained to deconvolve a scatter present in the electromagnetic signals (e.g., to created generated images 108). Signals having different wavelengths (e.g., within the NIR and SWIR range) and/or other characteristics (e.g., angle, polarization, frequency) propagate through human tissue and other media differently based on their respective wavelength and the refractive index of the media. The refractive index of the media through which the signals described herein propagate may follow deterministic patterns and modulations that can be learned by a machine learning model. The machine learning models disclosed herein can be trained to generate images of and/or identify features beneath tissue surfaces (e.g., up to at least 2 centimeters (cm) depth). Different layers of tissue may have different refractive indices. One or more machine learning models may be optimized for different layers of tissue by a plurality of respective models based on images obtained from different layers (e.g., using electromagnetic signals at different wavelengths).

For instance, the signals disclosed herein may be emitted into the human body. The human body is composed of about 60% water. Blood is made up of approximately 80% water, while cells contain around 70%. Collagen, the most abundant protein in the body, accounts for roughly 30% of the total protein content. When examining the different layers of tissue beneath the skin, down to a depth of about 2 cm, the anatomy can be described in terms of several distinct layers. An exemplary breakdown of the properties of such layers of tissue is provided below.

| | | Real part of the | |
|---|---|---|---|
| | | refractive index | |
| | | RI(λ), λ ∈ | RI(λ), λ ∈ |
| Structure | Depth | [400, 700] | [800, 1500] |
| Epidermis | 0.05 millimeters | ~1.50-1.56 | ~1.44-1.47 |
| | (mm) - 1.5 | | |
| | millimeters (mm) | | |
| Dermis | 1 millimeters (mm) - | ~1.37-1.45 | ~1.38-1.44 |
| | 1.4 millimeters (mm) | | |
| Hypodermis | 2 millimeters (mm) - | ~1.38-1.44 | ~1.37-1.44 |
| | xx* centimeters (cm) | | |
| | *depth may vary | | |
| Fascia | 1.5 centimeters | ~1.38-1.41 | ~1.37-1.40 |
| | (cm) - 2.0 | | |
| | centimeters (cm) | | |

The epidermis is the outermost layer of the skin, primarily made up of keratinized cells. The epidermis provides a waterproof barrier and creates skin tone. It has several sub-layers (like the stratum corneum and stratum basale), but overall, it is relatively thin. The dermis is directly beneath the epidermis and is a thicker layer of connective tissue that contains collagen and elastin fibers, providing the skin's elasticity and strength. The dermis supports the epidermis, provides sensation, and helps regulate temperature through sweat glands and blood flow. It houses important structures such as:
i. Blood vessels: For nutrient and oxygen supply.
ii. Nerves: For sensation.
iii. Hair follicles.
iv. Sweat glands and sebaceous (oil) glands.

The hypodermis begins at a depth of about 2 mm and can extend several centimeters, depending on the part of the body and an individual's fat distribution. This layer consists mainly of fat (adipose tissue) and loose connective tissue. It serves as a cushion and energy reserve and helps insulate the body. It also contains larger blood vessels and nerves. Its functionality includes shock absorption, insulation, and energy storage. Finally, the fascia is typically found at around 1.5 to 2.0 cm depth. The fascia is a tough layer of connective tissue that surrounds muscles, blood vessels, and nerves, holding them together and separating them from the skin and fat. The fascia provides support and structure, allowing muscles and other tissues to glide smoothly against one another.

One or more machine learning models may be trained to associate the obtained images 104 with ground-truth images 112 (e.g., fluorescent images, MRI images, ultrasound images) of the same layer of the tissue. The one or more machine learning models may be trained to predict structural features below the surface of the tissue at the respective layer based on the obtained images 104 and the one or more ground truth images 112 depicting that layer. Different machine learning models may be trained to identify features of interest within respective layers of the tissue. In some examples, each of the one or more machine learning models may generate a high-resolution (e.g., 1 micrometer resolution) image of a respective layer of the tissue, and the output of one or more of the machine learning models may be merged to generate a high-resolution image of a plurality of layers of the tissue.

Additionally, or alternatively, to training one or more machine learning models optimized for particular layers of tissue, in some examples, the one or more machine learning models may be trained as specialized models optimized for obtaining subsurface images from a sensor at the surface of a tissue and identifying features at one or more subsurface depths for any one or more of: a particular patient population (e.g., according to sex, age, body composition, etc.); a particular organ; a particular disease state, a particular tissue structure or microstructure, a particular spectral response associated with a particular type of structure or microstructure, a particular imaging device, particular wavelengths of electromagnetic signals emitted from an imaging device, etc. For example, one or more machine learning models may be trained based on images obtained using a first wavelength or set of wavelengths (e.g., in the NIR spectrum) and one or more machine learning models may be trained based on images obtained using a second wavelength or set of wavelengths (e.g., in the SWIR spectrum). As another example, one or more machine learning models may be trained to identify protein accumulations while one or more other machine learning models may be trained to identify lipid accumulations.

In some examples, during the ML-based processing stage 106, the one or more machine learning models are trained (e.g., via backpropagation) to learn optimized imaging settings, including illumination strength, illumination parameters (e.g. angle, polarization, frequency, target depth, polarization setup, focus settings, etc.), and/or microwave emissions from an optical phase clearer, to reduce (e.g., minimize) electromagnetic signal scattering. The one or more machine learning models may be trained to associate different imaging settings with respective amounts of scattering in different regions of interest below a surface of a live human tissue (e.g., at different depths, different tissue densities, etc.). The one or more machine learning models may be trained to determine imaging settings that reduce (e.g., minimize) a scattering of the electromagnetic signals obtainable by the sensor using a microwave phase array positioned at the surface of the live human tissue. The one or more machine learning models may be trained to determine least one of an illumination strength and an illumination parameter (e.g. angle, polarization, frequency, target depth, polarization setup, focus settings, etc.) of the one or more emitters that reduce (e.g., minimize) a scattering of the electromagnetic signals obtainable by the sensor. The one or more machine learning models may be trained to detect which region of tissue is being imaged and determine optimal imaging settings (e.g., imaging settings that minimize scattering) based on the detected region. Default imaging settings may be learned for different regions of interest in the body to facilitate efficient configuration of imaging settings. The system 100A may automatically adjust, in real time, one or more imaging settings to modify scattering based on the optimal settings determined using the one or more machine learning models.

In some examples, during the ML-based processing stage 106, the one or more machine learning models are trained to predict a disease state based on the identified feature(s) of interest 110. The feature(s) of interest 110 may be any subsurface structure or microstructure, including any one or more of a protein accumulation below the surface of the tissue, a lipid accumulation below the surface of the tissue, a spectral response of a tissue to the electromagnetic signals, nerves, blood vessels, glands, and/or lymph nodes and other structures of the lymphatic system, etc. The disease state may include cardiac amyloidosis, atherosclerosis, carpal tunnel syndrome, other forms of amyloidosis (e.g., light chain amyloidosis), peripheral neuropathies (e.g. caused by diabetes), or other disease states detectable based on micron-scale features of interest beneath the surface of a tissue.

In some examples, during the ML-based processing stage 106, the one or more machine learning models are trained to generate embeddings based on the obtained images 104. Embeddings are low-dimensional representations that capture detailed semantic information of the images. For instance, the embeddings may include morphological information associated with different biological structures, spectral information associated with different biological structures, and/or other features included in the images. In some examples, the embeddings may capture a time dimension associated with time-series image data such that the embeddings enable analysis of time-variant characteristics of the images.

During an inferencing phase of ML-based processing stage 106, The obtained images 104 may be input into one or more trained machine learning models. For instance, as discussed above, a sensor may be positioned at the surface of a tissue and electromagnetic signals reflected from or transmitted through live human tissues of different densities may be obtained using the sensor as one or more obtained images. The one or more trained machine learning models may create high-resolution images (e.g., 1 micrometer resolution images) based on obtained images 104. The one or more trained machine learning models may identify features of interest, including but not limited to structures and/or microstructures such as protein accumulations, lipid accumulations, inflamed nerves, lymphatic structures, spectral responses of different tissues to the electromagnetic signals, and other structures, microstructures, etc. beneath the surface of a tissue, based on the obtained images. The one or more trained machine learning models may predict disease state(s) based on the features of interest. The one or more trained machine learning models may model disease progressions based on the features of interest.

The one or more trained machine learning models may determine optimized imaging settings to reduce (e.g., minimize) scattering of electromagnetic signals as they propagate within the tissue. For instance, the one or more obtained images 104 may be input into the one or more machine learning models, and the one or more machine learning models may determine one or more imaging settings to reduce scattering of the electromagnetic signals to improve an image quality. The one or more trained machine learning models may determine the one or more imaging settings based at least in part on a detected region and/or layer of tissue being imaged. The one or more imaging settings may include microwave emissions from an optical phase clearer, illumination strength, and/or illumination parameters (e.g. angle, polarization, frequency, target depth, polarization setup, focus settings, etc.). The system 100A may adjust the imaging settings, optionally in real time, based on the determined settings output by the one or more machine learning models.

In some examples, the one or more trained machine learning models may be used to identify scattering of certain electromagnetic signals comprising each of the one or more obtained images 104, correct for the scattering to create one or more generated images 108 of tissues at one or more subsurface depths, and/or identify features of interest 110 within the one or more images of tissues at the one or more subsurface depths. The one or more trained machine learning models may predict a disease state based on the identified micron-scale feature of interest. The features of interest and/or disease state may be or include any of those described above.

FIG. 1B illustrates an exemplary system 100B for identifying features of interest (e.g., micron-scale features of interest) beneath the surface of a tissue that may include or be used for aspects of system 100A. System 100B includes at least one imaging device 120. Imaging device 120 may include one or more emitters 122, including any of the emitters described with reference to system 100A. Imaging device 120 may include one or more sensors 124, such as any of the sensors described with reference to system 100A. Imaging device 120 may include one or more optical phase clearers (OPC) 126, such as any of the optical phase clearers described with reference to system 100A. Imaging device 120 may include one or more diffractive optical elements (DOE) 128 and/or one or more Metalense 130. The DOE 128 and/or Metalense 130 may be configured to modify a characteristic/property of light or other electromagnetic signals emitted from the one or more emitters and/or modify a characteristic/property of the light or other electromagnetic signals received by the one or more sensors. The DOE 128 and/or Metalense 130 may modify the polarization of light or other electromagnetic signals emitted from the one or more emitters 122, modify the generation of a dot matrix by the one or more emitters 122, and/or modify the depth of a beam generated by the one or more emitters 122. The DOE 128 and/or Metalense 130 may modify the polarization of light or other electromagnetic signals received by the one or more sensors 124 and/or modify the depth of a beam received by the one or more sensors 124.

The at least one imaging device 120 may include a memory 132 and one or more processors 134. The memory 132 may store one or more training programs (e.g., machine learning algorithms) for training one or more machine learning models according to any of the methods disclosed herein. The memory 132 may store one or more trained machine learning models trained to perform any of the inferencing tasks disclosed herein. The one or more processors 134 may be configured to execute the one or more training programs and/or one or more machine learning models to process input data, including images and/or electromagnetic signals obtained using the one or more sensors 124. The one or more processors 134 may be configured to control adjustable settings of the one or more emitters 122, one or more sensors 124, OPC 126, DOE 128, and/or Metalense 130. The imaging device may include a display 138 (e.g., for displaying images and/or outputs of the one or more machine learning models). In some examples, the memory 132 and the one or more processors 134 may be configured to perform one or more aspects of the methods disclosed herein, such as detection of signals/images, and may transmit data to one or more other electronic devices for further analysis. The imaging device 120 may include a communications unit 136. The communications unit 136 may be configured to transmit data to one or more local devices 140 (e.g., for local processing using additional compute resources) and/or one or more remote servers 150 (e.g., for processing on the cloud).

The one or more local devices 140 may be connected to the imaging device 120 using one or more wired or wireless electronic communication protocols, such as WiFi and/or Bluetooth. The one or more local devices 140 may include nearby hardware devices (e.g., devices located within a local area network, within Bluetooth range, etc.), such as a mobile phone or PC. The one or more local devices 140 may include a memory 142, one or more processors 146, a display 144, and/or a communications unit 148 (e.g., for receiving data from and transmitting data to imaging device 120). The memory 142 may store one or more training programs (e.g., machine learning algorithms) for training one or more machine learning models according to any of the methods disclosed herein. The memory 142 may store one or more trained machine learning models trained to perform any of the inferencing tasks disclosed herein. The memory 142 may include relatively more storage capacity than memory 132 of imaging device 120. The one or more processors 146 may have more processing capabilities relative to the one or more processors 124 of imaging device 120. Accordingly, more complex analysis of the data collected using imaging device 120 may be performed at the one or more local devices 140. For instance, the one or more local devices may input images and/or electromagnetic signals detected using imaging device 120 into one or more machine learning models to detect features of interest, generate high-resolution images, predict disease states, or perform any other processing task disclosed herein.

The one or more remote servers 150 may be connected to the imaging device 120 using one or more wired or wireless electronic communication protocols. The one or more remote servers 150 may be configured to operate a cloud computing environment. The one or more remote servers 150 may include a memory 152, one or more processors 156, a display 154, and/or a communications unit 158 (e.g., for receiving data from and transmitting data to imaging device 120). The memory 152 may store one or more training programs (e.g., machine learning algorithms) for training one or more machine learning models according to any of the methods disclosed herein. The memory 152 may store one or more trained machine learning models trained to perform any of the inferencing tasks disclosed herein. The memory 152 may include relatively more storage capacity than memory 132 of imaging device 120 and/or memory 142 of local device 140. The one or more processors 154 may have more processing capabilities relative to the one or more processors 124 of imaging device 120 and/or the one or more processors 146 of local device(s) 140. Accordingly, more complex analysis of the data collected using imaging device 120 may be performed at the one or more remote servers 150. For instance, the one or more local devices may input images and/or electromagnetic signals detected using imaging device 120 into one or more machine learning models to detect features of interest, generate high-resolution images, predict disease states, or perform any other processing task disclosed herein.

It should be understood that any of the various methods disclosed herein (e.g., processing of images and/or electromagnetic signals using one or more machine learning models, training one or more machine learning models, etc.) may be performed using compute resources of the imaging device used to obtain the electromagnetic signals and/or images, a local computing resource capable of receiving data from the imaging device (e.g., a mobile phone, laptop, etc.), a cloud computing platform, or any combination thereof. In some examples system 100A and/or 100B may utilize processing capabilities of a plurality of different devices to perform any of the methods disclosed herein. In some examples, processing tasks may instead be performed using only the compute resources provided on a wearable device or other portable imaging device. Thus, the one or more models developed for training and inference with respect to the image reconstruction capabilities, feature detection capabilities, and other capabilities disclosed herein, may be resident on the imaging device (e.g., wearable device, endoscope, or other imaging device), on a nearby mobile device or PC, in a cloud computing platform, or a combination of two or more of these.

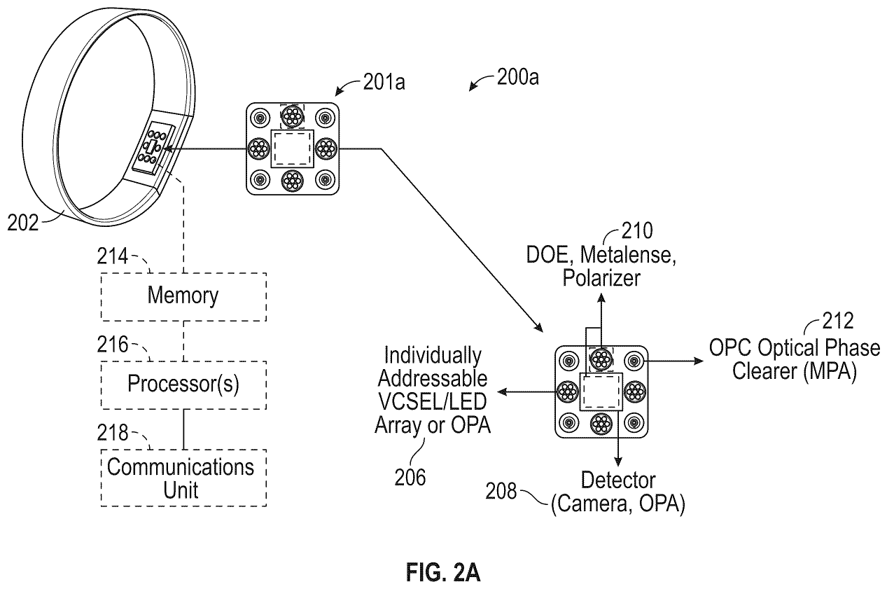

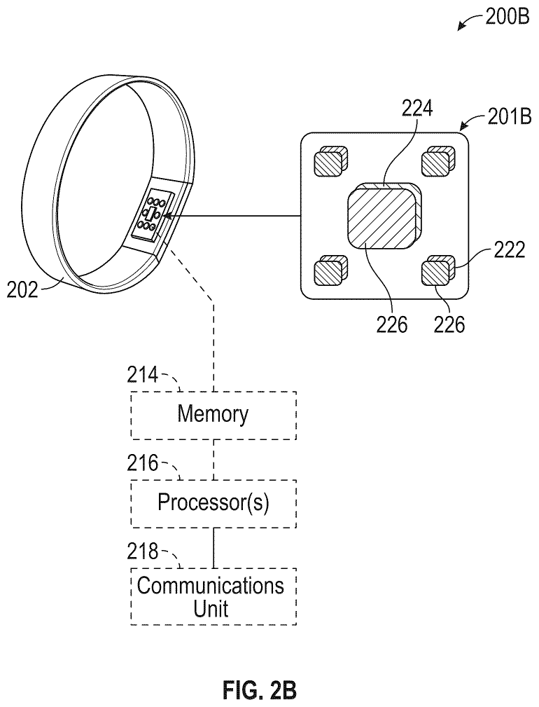

FIG. 2A illustrates an exemplary device 200A for identifying features of interest (e.g., micron-scale features of interest) beneath the surface of a tissue that may include or be used for aspects of system 100A and/or 100B described above. Device 200A includes an imaging device 201A. Imaging device 201A may be provided on a wearable device, such as wristband 202. Imaging device 201A may include one or more emitters 206. The emitters 206 may include one or more coherent light sources, such as light emitting diodes (LED) or vertical-cavity surface-emitting lasers (VCSEL). The VCSEL and/or LEDs may be individually addressable. The emitters 206 may include one or more optical phase arrays (OPA) in the infrared spectrum. The emitters 206 may be configured to the emit electromagnetic signals comprised of near infrared or short-wave infrared light.

Imaging device 201A may include one or more sensors 208. The sensors 208 may include a camera configured to capture the near infrared spectrum and the short-wave infrared spectrum. The camera may be configured to capture at least part of the visible spectrum. The sensors 208 may include an optical phase array configured to capture the near infrared spectrum and/or the short-wave infrared spectrum. The imaging device 201A may also include one or more electromagnetic signal manipulation devices 210. The one or more electromagnetic signal manipulation devices 210 may include one or more diffractive optical elements (DOE) and/or one or more Metalenses. The one or more electromagnetic signal manipulation devices 210 may be positioned on the imaging device 201A such that they can modify properties/characteristics of the light or other electromagnetic signals emitted from the one or more emitters 206 and/or the light or other electromagnetic signals received by the one or more sensors 208. The one or more electromagnetic signal manipulation devices 210 may modify the polarization of light or other electromagnetic signal emitted from the one or more emitters 206, modify the generation of a dot matrix by the one or more emitters 206, and/or modify the depth of a beam generated by the one or more emitters 206. The one or more electromagnetic signal manipulation devices 210 may modify the polarization of light or other electromagnetic signals received by the one or more sensors 208 and/or modify the depth of a beam received by the one or more sensors 208.

Imaging device 201A may include one or more optical phase clearers 212. The one or more optical phase clearers 212 may include one or more optical phase arrays in the microwave region (Microwave Phase Array (MPA)). The imaging device 201A, or aspects thereof, may be programmable such that one or more imaging settings, including microwave emissions, illumination strength, and/or illumination parameters (e.g. angle, polarization, frequency, target depth, polarization setup, focus settings, etc.) can be configured, optionally in real time, to optimize imaging conditions.

Device 200A may include one or more processors 216 and a memory 214. The memory 214 may store one or more machine learning models that may be trained and/or used for inferencing tasks according to various aspects of this disclosure. Images acquired using imaging device 201 may be input into one or more machine learning models stored in a memory 214 of device 200A to create one or more images of live human tissues at one or more subsurface tissue depths, identify micron-scale features of interest within one or more images of live human tissues at the one or more subsurface tissue depths, predict disease states, determine optimized imaging settings, and/or other tasks as described in additional detail throughout. Device 200A may also include a communications unit 218 for transmitting data to and receiving data from one or more other computing devices/systems. For instance, as discussed with reference to FIG. 1B, some processing may be performed at an imaging device such as imaging device 200A and some processing may be performed at another local computing device (e.g., a mobile phone or laptop) and/or using a cloud computing platform.

FIG. 2B illustrates another exemplary device 200B for identifying features of interest (e.g., micron-scale features of interest) beneath the surface of a tissue that may include or be used for aspects of system 100A, 100B, and/or 200A described above. Device 200B includes an imaging device 201B. Imaging device 201B may be provided on a wearable device, such as wristband 202. Imaging device 201B may include one or more emitters 222. The emitters 222 may include one or more coherent light sources, such as light emitting diodes (LED) or vertical-cavity surface-emitting lasers (VCSEL). The emitters 222 may include one or more optical phase arrays (OPA) in the infrared spectrum. The emitters 222 may be configured to the emit electromagnetic signals comprised of near infrared or short-wave infrared light.

Imaging device 201B may include one or more sensors 224. The sensors 224 may include a camera configured to capture the near infrared spectrum and the short-wave infrared spectrum. The camera may be configured to capture at least part of the visible spectrum. The sensors 224 may include an optical phase array configured to capture the near infrared spectrum and/or the short-wave infrared spectrum. The imaging device 201B may also include one or more light manipulation devices 226. The one or more light manipulation devices 226 may include one or more diffractive optical elements (DOE) and/or one or more Metalenses. The one or more light manipulation devices 226 may be positioned on the imaging device 201B such that they can modify properties/characteristics of the light emitted from the one or more emitters 222 and/or the light received by the one or more sensors 224. The one or more light manipulation devices 226 may modify the polarization of light emitted from the one or more emitters 222, modify the generation of a dot matrix by the one or more emitters 222, and/or modify the depth of a beam generated by the one or more emitters 222. The one or more light manipulation devices 226 may modify the polarization of light received by the one or more sensors 224 and/or modify the depth of a beam received by the one or more sensors 224. The imaging device 201B, or any aspects thereof, may be programmable such that one or more imaging settings, including microwave emissions, illumination strength, and/or illumination parameters (e.g. angle, polarization, frequency, target depth, polarization setup, focus settings, diffraction patterns etc.) can be configured, optionally in real time, to optimize imaging conditions.

Device 200B may, like device 200A, include one or more processors 216 and a memory 214. The memory 214 may store one or more machine learning models that may be trained and/or used for inferencing tasks according to various aspects of this disclosure. Images acquired using imaging device 201B may be input into one or more machine learning models stored in a memory 214 of device 200B to create one or more images of live human tissues at one or more subsurface tissue depths, identify micron-scale features of interest within one or more images of live human tissues at the one or more subsurface tissue depths, predict disease states, determine optimized imaging settings, and/or other tasks as described in additional detail throughout. Device 200B may also include communications unit 218 for transmitting data to and receiving data from one or more other computing devices/systems. For instance, as discussed with reference to FIG. 1B, some processing may be performed at an imaging device such as imaging device 200B and some processing may be performed at another local computing device (e.g., a mobile phone or laptop) and/or using a cloud computing platform.

### Exemplary Method for Training a Machine Learning Model

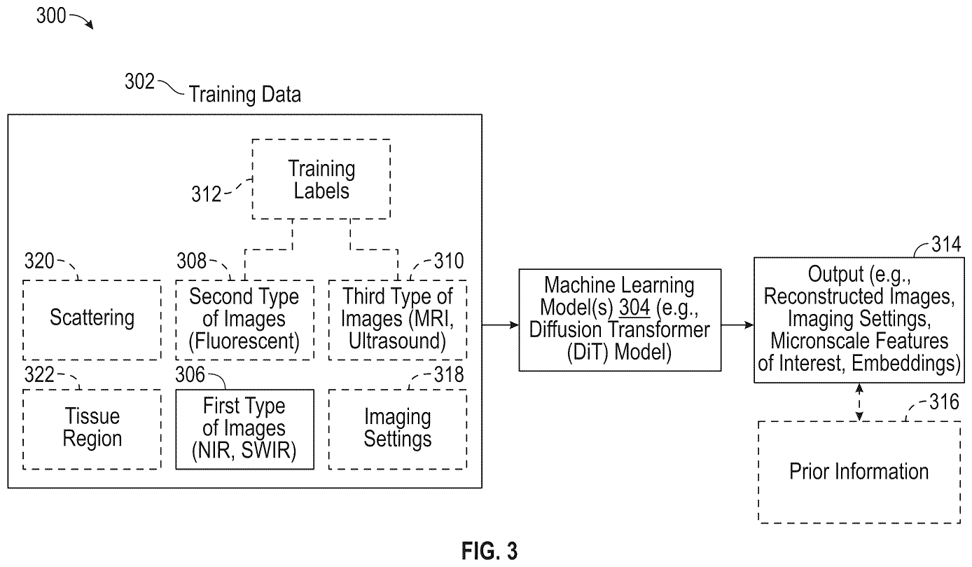

FIG. 3 illustrates an exemplary process 300 for training one or more machine learning models 304. The one or more machine learning models 304 may be trained to create one or more images of live human tissues at one or more subsurface tissue depths. The one or more machine learning models 304 may be trained to identify micron-scale features (e.g., as small as 1 micron) of interest within one or more images of live human tissues at the one or more subsurface tissue depths. The one or more machine learning models 304 may be trained to determine imaging settings 318 to optimize (e.g., reduce, minimize) scattering of electromagnetic signals used to obtain images of live human tissues at the one or more subsurface tissue depths. The one or more machine learning models may be trained to reconstruct a three-dimensional model of an anatomy included below the surface of a live human tissue.

The one or more machine learning models 304 may include a Diffusion Transformer (DiT) Model, similar to the DiT model described in William Peebles, Saining Xie, Scalable Diffusion Models with Transformers, https://arxiv.org/abs/2212.09748 (2023), which is incorporated herein by reference in its entirety. The DiT model may include aspects of diffusion model architecture and transformer model architecture, enabling the model to leverage the self-attention and cross-attention mechanisms of the transformer architecture with the denoising capabilities of diffusion models.

With reference to FIG. 3, training data 302 may include a first type of images 306, a second type of images 308, and/or a third type of images 310. In some examples, the first type of images 306, the second type of images 308, and the third type of images 310 include the image data of the same region of human tissue. Alignment between the first type of images 306, the second type of images 308, and the third type of images 310 may be achieved with reference to biological candles. Biological candles refer to biological landmarks that are assumed to be stationary within the body. At least a subset of the training images of the second type and/or the third type may be labeled to indicate any of: a disease state, a tissue classification, a biomarker, a segmentation mask of a disease feature, a protein accumulation, a lipid accumulation, and a physiological parameter.

In some examples, the first type of images 306 include label free images obtained by exposing a live human tissue to near infrared (NIR) or short-wave infrared (SWIR) light (e.g., using aspects of system 100A, system 100B, device 200A and/or device 200B described above). The first type of images 306 may have a dimensionality of (H₀, W₀, D₀, T₀, P₀) in which H indicates the height, W indicates the width, D indicates the depth (into the tissue), T indicates a time index, and P indicates the imaging settings (including illumination strength, illumination parameters, and/or microwave emissions). The time index may be included when the first type of images includes time-series images. Time-series images may be used to train the machine learning model to learn time variant features, such as disease progression, morphological changes, etc.

In some examples the first type of images 306 are obtained by emitting, from one or more emitters at the surface of the live human tissue, electromagnetic signals capable of penetrating human tissue of different densities and obtaining from a sensor positioned at the surface of the live human tissue electromagnetic signals reflected from or transmitted through live human tissues of different densities as one or more obtained images. As discussed with reference to FIG. 1A, different regions of the live human tissue may have different refractive indices, resulting in varied scattering of the electromagnetic signals at different tissue regions (e.g., different depths).

In some examples, electromagnetic signal scattering is modeled by a set of stochastic processes from a family of exponential functions (e.g. gaussian, poisson) with unknown kernels. One or more machine learning models 304 can be trained to learn and correct for the scattering to generate images and identify features in the images. For instance, the one or more machine learning models 304 can be trained to deconvolve electromagnetic signals to generate images of structures including nerves, blood vessels, glands, lymph nodes and other structures of the lymphatic system, etc. The one or more machine learning models 304 can also be trained to predict disease states, model disease progressions and/or morphological changes, etc.

In some examples, the second type of image(s) 308 includes fluorescent image(s) of a biological sample (live or fixed) or 3D local properties of the sample (e.g. complex refractive index). The fluorescent image(s) of the biological sample may be stained with indocyanine green dye (or alternative fluorophore) to label biological structures (e.g., cardiovascular structures, lymphatic structures, nervous system structures) or other parts of the sample. In some examples, fluorescent image(s) are obtained using one or more of the same imaging settings 318, including optical phase clearer settings, illumination strength, and/or illumination parameters, as used for the first type of images 306. In some examples, one or more clinically approved dies are used to enhance the transparency of the biological sample. The fluorescent image(s) may be acquired with a widefield or confocal setup at relatively high axial and lateral resolution. The acquisition of the second type of image(s) generates a cube of (H₁, W₁, D₁) dimension with N₁=H₁*W₁*D₁ unknowns to solve.

In some examples, the third type of images 310 include MRI and/or ultrasound images. The data cube generated by acquisition of the third type of images 310 may be N₂=H₂*W₂*H₂. N₁ (corresponding to the second type of images 308) and N₂ are not necessarily equal. The second type of images 308 and/or the third type of images 310 may be paired with training labels 312. The training labels 312 may include disease state indicators, classifications of tissues, biomarkers, a segmentation mask of a disease feature, a protein accumulation, a lipid accumulation, and a physiological parameter (e.g., temperature, blood pressure, heart rate, nerve conductivity), etc. In some examples, the second type of images 308 and/or the third type of images 310 include images of known protein accumulations and/or lipid accumulations. The training labels 312 may include indications of such known protein accumulations and/or lipid accumulations and may be paired with such images.

The training data 302 may include imaging settings 318 used to obtain the first type of images 306. The imaging settings may include settings for an optical phase clearer (e.g., microwave emissions), illumination strength of one or more emitters, and/or other illumination parameters (e.g. angle, polarization, frequency, target depth, polarization setup, focus settings, etc.). The imaging settings may be associated in the training data with scattering 320 (e.g., an amount of scattering associated with those imaging settings) and/or one or more tissue regions 322. For instance, imaging settings may be associated with a different amount of scattering at a first tissue depth and/or density than at a second tissue depth and/or density. The one or more machine learning models 304 can thus be trained to learn associations between imaging settings 318 and different amounts of electromagnetic signal scattering 320 at different tissue regions 322.

The training data 302 can be used to train one or more machine learning models 304. The first type of images 306, second type of images 308, and/or third type of images 310 may be input into machine learning model (or first module of a machine learning model) 304. Optionally, any of the images may be preprocessed (e.g., normalized, cropped, rotated, etc.) prior to receipt by machine learning model 304. During training, the one or more machine learning models 304 (or a module of machine learning model 304) may be trained to generate an output 314. Machine learning model 304 may be trained to reconstruct from first type of images 306, images of known structures and/or microstructures, including any one or more of protein accumulations, lipid accumulations, lymphatic structures, nervous system structures, etc., included in the second type of images 308 and/or the third type of images 310. The one or more machine learning models 304 (or a module of machine learning model 304) may additionally, or alternatively, be trained to identify features of interest (e.g., micron-scale features of interest), such as protein accumulations and/or lipid accumulations within the one or more images reconstructed images. In some examples, the one or more machine learning models 304 may trained to identify one or more features of interest (e.g., micron-scale features of interest) based on embedding representations of the input images. In some examples, the one or more machine learning models 304 (or a module of machine learning model 304) may be trained to reconstruct the three-dimensional model of an anatomy below the surface of the tissue.

In some examples, the generated images and/or three-dimensional model of an anatomy below the surface of the tissue are compared to prior information 316, such as a ground-truth three-dimensional model of an anatomy below the surface of the tissue, defining various anatomical characteristics of different tissue types in a sample. A ground-truth model defining expected physical attributes of the anatomy can be used to evaluate the performance of the system to detect (segment) known anatomical features (e.g. median nerve, ulnar arteries, etc.).

In some examples, the machine learning model 304 (or a module of machine learning model 304) may be trained to determine optimizes imaging settings of one or more emitters (e.g., illumination strength and/or illumination parameters) and/or an optical phase clearer (e.g., a microwave emission) to modify scattering in electromagnetic signals that comprise the first type of images 306. Imaging devices such as those described above with reference to system 100A, system 100B, device 200A, and/or device 200B be adjustable such that images (e.g., NIR and SWIR images) may be obtained using a plurality of different imaging settings. For instance, system 100A, system 100B, device 200A, and/or device 200B may include a programmable illumination source (e.g., LED, laser) and/or the programmable optical phase clearer. Training the machine learning model(s) described herein may include training the models to learn optimal imaging settings 318 to mitigate/modify signal scattering for different imaging scenarios (e.g., different tissue regions, tissues at different depths and/or densities, different imaging devices, etc.). An exemplary system (e.g., system 100A and/or system 100B) may automatically configure the programmable imaging devices according to the determined optimal settings to mitigate scattering.

In some examples, the one or more machine learning models 304 may be trained to generate embeddings based on the first type of images 306, second type of images 308, and/or third type of images 310. Images may be split into patches and each patch may be encoded into an embedding vector. Embeddings are low-dimensional representations that capture detailed semantic information of the images. For instance, the embeddings may include morphological information associated with different biological structures, spectral information associated with different biological structures, and/or other features included in the images. In some examples, the embeddings may capture a time dimension associated with time-series image data such that the embeddings enable analysis of time-variant characteristics of the images. The one or more machine learning models 304 can thus learn to capture rich semantic information from each image in the form of an embedding. In some examples, the one or more machine learning models 304 may be trained to reconstruct from embeddings of first type of images 306, images of known protein accumulations or lipid accumulations included in the second type of images 308 and/or the third type of images 310. In some examples, the embeddings obtained from the first type of images 306 may be used for downstream analyses, such as detection of disease states, modeling of disease progression and/or morphological changes, etc.

Process 300 is performed, for example, using one or more electronic devices implementing a software platform. In some embodiments, process 300 is performed using any or all of the aspects of system 100A, system 100B, device 200A, and/or device 200B described above with reference to FIGS. 1A, 1B, 2A, and 2B. In some embodiments, process 300 is performed using a client-server system, and the blocks of process 300 are divided up in any manner between the server and one or more client devices. Thus, while portions of process 300 are described herein as being performed by particular devices, it will be appreciated that process 300 is not so limited. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail above) are exemplary by nature and, as such, should not be viewed as limiting.

### Exemplary Method for Training a Machine Learning Model to Identify Micron-Scale Features

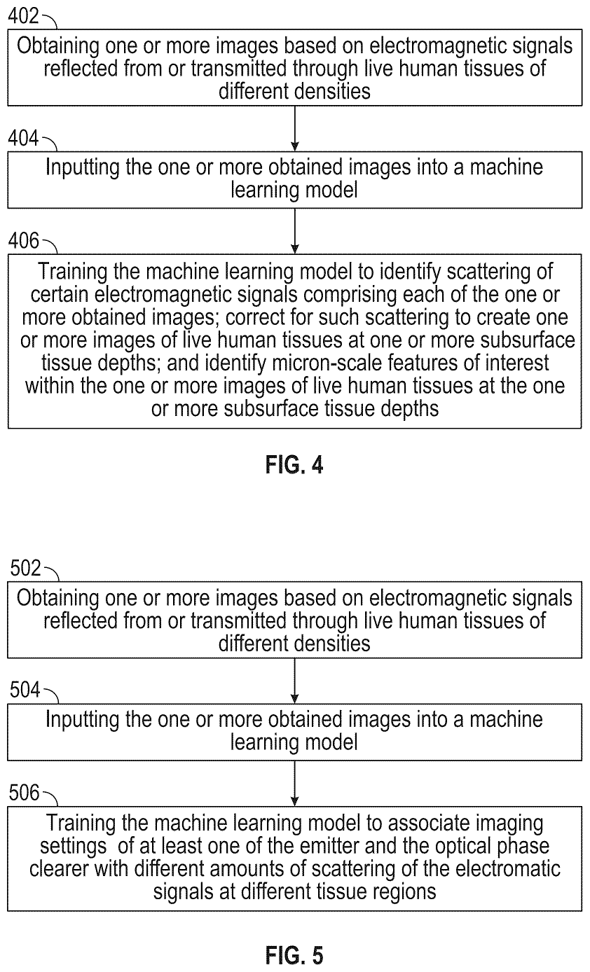

FIG. 4 illustrates an exemplary process 400 for training a machine learning model to identify micron-scale features based on images of live human tissues. Process 400 may share one or more features in common with the training process described with reference to FIG. 3. Process 400 includes obtaining one or more images based on electromagnetic signals reflected from or transmitted through live human tissues of different densities, inputting the one or more obtained images into a machine learning model, and training the machine learning model to identify scattering of certain electromagnetic signals comprising each of the one or more obtained images, correct for such scattering to create one or more images of live human tissues at one or more subsurface tissue depths, and identify micron-scale features of interest within the one or more images of live human tissues at the one or more subsurface tissue depths.

At block 402, an exemplary system obtains one or more images based on electromagnetic signals reflected from or transmitted through live human tissues of different densities. The electromagnetic signals may be comprised of near infrared or short-wave infrared light. Obtaining the one or more images may include emitting from one or more emitters at the surface of the live human tissue electromagnetic signals capable of penetrating human tissue of different densities and obtaining from a sensor positioned at the surface of the live human tissue electromagnetic signals reflected from or transmitted through live human tissues of different densities as the one or more obtained images.

Obtaining the one or more images may involve adjusting at least one of an illumination strength and an illumination parameter of the one or more emitters to modify a scattering of the electromagnetic signals obtainable by the sensor. The illumination parameter may include any one or more of an illumination angle, a polarization, a frequency, and a target depth. Obtaining the one or more images may include modifying a scattering of the electromagnetic signals obtainable by the sensor using a microwave phase array positioned at the surface of the live human tissue. The illumination strength, illumination parameter(s), and/or microwave phase array may be adjustable in real time (e.g., using one or more machine learning models trained to optimize imaging settings to mitigate (e.g., minimize) signal scattering).

In some examples, the one or more emitters include one or more coherent light sources. In some examples, the one or more emitters include an optical phase array. The sensor the sensor may include a camera configured to capture the near infrared spectrum and the short-wave infrared spectrum. The camera may be configured to capture at least part of the visible spectrum. The sensor may include an optical phase array configured to capture the near infrared spectrum and the short-wave infrared spectrum.

At block 404, the exemplary system inputs the one or more obtained images into the machine learning model (e.g., a diffusion transformer model, such as the machine learning model(s) described with reference to FIG. 3). At block 406, the exemplary system may train the machine learning model to identify scattering of certain electromagnetic signals comprising each of the one or more obtained images, correct for such scattering to create one or more images of live human tissues at one or more subsurface tissue depths, and identify micron-scale features of interest within the one or more images of live human tissues at the one or more subsurface tissue depths. The micron scale feature of interest may be a protein accumulation, a lipid accumulation, and/or a spectral response of a tissue to the electromagnetic signals.

Training the machine learning model may include associating training images of known protein accumulations or lipid accumulations with subdermal training images obtained from a sensor positioned at the surface of a live human tissue. Training the machine learning model may include training the machine learning model to reconstruct training images of known protein accumulations or lipid accumulations. The training images of known protein accumulations or lipid accumulations may include any one or more of a fluorescent image, a local refractive index image, a magnetic resonance imaging (MRI) image, and an ultrasound image. The training images may include a training image of a first type depicting a region of a live human tissue and a training image of a second type depicting the same region of the live human tissue. At least a subset of the training images may be labeled to indicate any one or more of a disease state, a tissue classification, a biomarker, a protein accumulation, and a lipid accumulation.

Training the machine learning model may include training the machine learning model to reconstruct from the obtained or created images a three-dimensional model of an anatomy below the surface of the tissue. Training the machine learning model may include training the machine learning model to deconvolve a scatter present in the electromagnetic signals. The one or more obtained images may include a plurality of images, and the machine learning model may be trained to correct for the scattering to create a plurality of images of live human tissues at a plurality of subsurface tissue depths.

Process 400 is performed, for example, using one or more electronic devices implementing a software platform. In some embodiments, process 400 is performed using any or all of the aspects of system 100A, system 100B, device 200A, and/or device 200B described above with reference to FIGS. 1A, 1B, 2A and 2B. In some embodiments, process 400 is performed using a client-server system, and the blocks of process 400 are divided up in any manner between the server and one or more client devices. Thus, while portions of process 400 are described herein as being performed by particular devices, it will be appreciated that process 400 is not so limited. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail above) are exemplary by nature and, as such, should not be viewed as limiting.

Exemplary Method for Training a Machine Learning Model to Associate Imaging Settings with Scattering of Electromatic Signals at Different Tissue Regions

FIG. 5 illustrates an exemplary process 500 for training one or more machine learning models to associate imaging settings of at least one of the emitter and the optical phase clearer to with different amounts of scattering of the electromagnetic signals at different tissue regions (e.g., tissue depths). Process 500 may involve training a machine learning model to learn imaging settings including illumination strength, illumination parameters such as illumination angle, polarization, frequency, and target depth, and/or microwave emission from an optical phase clearer associated with different amounts of scattering of electromagnetic signals used to obtain images of tissue beneath a tissue surface.

At block 502, an exemplary system may obtain one or more images based on electromagnetic signals reflected from or transmitted through live human tissues of different densities. The electromagnetic signals may be comprised of near infrared or short-wave infrared light. The images may be obtained according to any of the steps described above. Obtaining the one or more images may include emitting from one or more emitters at the surface of the live human tissue electromagnetic signals capable of penetrating human tissue of different densities and obtaining from a sensor positioned at the surface of the live human tissue electromagnetic signals reflected from or transmitted through live human tissues of different densities as the one or more obtained images. An optical phase clearer (e.g., a microwave phase array (MPA)) may be used to modify scattering of the electromagnetic signals. At block 504, the exemplary system may input the one or more obtained images into one or more machine learning models (e.g., a diffusion transformer model, such as model 304 of FIG. 3).

At block 506, the system may train the one or more machine learning models to associate imaging settings of at least one of the emitter and the optical phase clearer with different amounts of scattering of the electromagnetic signals at different tissue regions. The imaging settings may include illumination strength, illumination parameters, and/or microwave emission. The one or more machine learning models may be trained to associate imaging settings with respective amounts of scattering in different regions of interest below a surface of a live human tissue (e.g., at different depths, different tissue densities, etc.). The one or more machine learning models may be trained to detect which region of tissue is being imaged and determine optimized imaging settings based on the detected region. For instance, the one or more machine learning models may be trained to detect biological candles and configure the imaging settings based on the detected biological candles. Default imaging settings may be learned for different regions of interest in the body (e.g., associated with different biological candles) to facilitate efficient configuration of imaging settings.

Process 500 is performed, for example, using one or more electronic devices implementing a software platform. In some embodiments, process 500 is performed using any or all of the aspects of system 100A, system 100B, device 200A, and/or device 200B described above with reference to FIGS. 1A, 1B, 2A, and 2B. In some embodiments, process 500 is performed using a client-server system, and the blocks of process 500 are divided up in any manner between the server and one or more client devices. Thus, while portions of process 500 are described herein as being performed by particular devices, it will be appreciated that process 500 is not so limited. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail above) are exemplary by nature and, as such, should not be viewed as limiting.

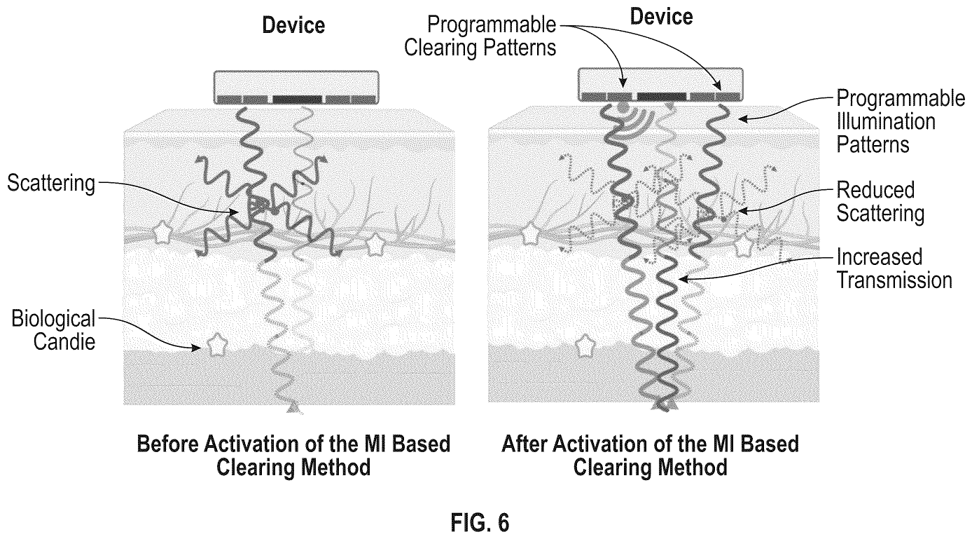

FIG. 6 depicts an illustrative representation of electromagnetic signal scattering before and after activation of a machine-learning based signal clearing method. Before activation of the machine-learning based signal clearing method scattering is significantly higher relative to scattering after activation of the learned imaging settings. After activation of the learned imaging settings, scattering is reduced and signal transmission is increased relative to before activation of the learned imaging settings. The region of tissue being imaged may be detected by the model based on detection of one or more biological candles, as discussed above with reference to FIG. 5. The machine learning model may automatically activate learned imaging settings associated with the detected biological candles.

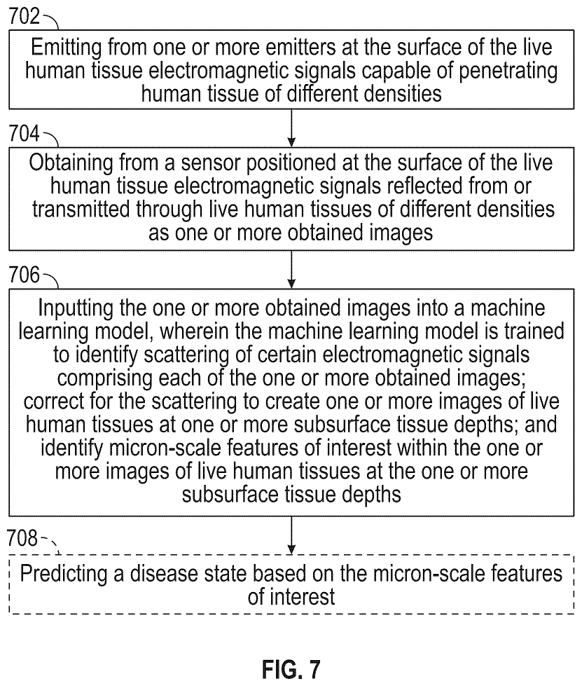

FIG. 7 illustrates an exemplary process 700 for identifying micron-scale features below a surface of a live human tissue. Process 700 includes emitting electromagnetic signals at the surface of a live human tissue and obtaining reflections of those signals (and/or signals transmitted through the tissue) using a sensor as images. The obtained images can be input into a machine learning model trained to identify scattering of certain electromagnetic signals, correct for the scattering to create one or more images of live human tissues at one or more subsurface tissue depths, and identify micron-scale features of interest within the one or more images of live human tissues at the one or more subsurface tissue depths.

At block 702, an exemplary system (e.g., system 100A, system 100B) emits, from one or more emitters at the surface of the live human tissue, electromagnetic signals capable of penetrating human tissue of different densities. The emitted electromagnetic signals may be comprised of near infrared (NIR) or short-wave infrared (SWIR) light. Signals having different wavelengths (e.g., within the NIR and SWIR range) propagate through human tissue (and other mediums) differently based on their respective wavelength and the refractive index of the tissue (e.g., as described above with reference to FIG. 1A). The electromagnetic signals may be emitted from one or more coherent light sources, such as a light emitting diode (LED) or a vertical-cavity surface-emitting laser (VCSEL). The electromagnetic signals may additionally, or alternatively, be emitted using one or more optical phase array(s) (OPA). The OPA may be capable of performing beam steering in the infrared region.

At block 704, the exemplary system obtains from a sensor positioned at the surface of the live human tissue, electromagnetic signals reflected from or transmitted through live human tissues of different densities as one or more obtained images. The sensor may be included on a wearable device, such as a ring, glasses, a patch, a wristband (e.g., as shown in FIGS. 2A and 2B), a watch, etc. The sensor may be included on a medical device, such as an endoscope, flex-scope, or other medical imaging device. The sensor may include a camera configured to capture the near infrared spectrum and the short-wave infrared spectrum. The camera may also be configured to capture at least part of the visible spectrum. The sensor may include an optical phase array configured to capture the near infrared spectrum and the short-wave infrared spectrum.

The system may actively optimize imaging settings (e.g., based upon characteristics of the obtained images) to reduce or otherwise modify scattering of the electromagnetic signals. Signal scattering depends, at least in part, on the illumination strength and the parameters of the illumination (e.g. angle, polarization, frequency, target depth). In some examples, the system automatically configures an illumination strength and/or an illumination parameter of the one or more emitters to modify a scattering of the electromagnetic signals obtainable by the sensor. In some examples, the system modifies a scattering of the electromagnetic signals obtainable by the sensor using one or more optical phase clearers (OPC) positioned at the surface of the live human tissue. The one or more optical phase clearers (OPC) may operate in the microwave region (e.g., a microwave phase array (MPA)). The system may configure illumination strength and parameters along with microwave emission from the MPA, by adjusting the illumination strength, illumination parameters, and microwave emission from the MPA in real time, to optimize image acquisition from specific regions of interest.

In some examples, one or more machine learning models (or one or more modules of a machine learning model) are trained to determine optimizes imaging conditions. An exemplary system (e.g., system 100A, system 100B) may adjust the illumination strength, illumination parameters, and microwave emission from the MPA, optionally in real time during imaging. The one or more machine learning models may be trained to associate imaging settings including illumination strength, illumination parameters, and/or microwave emission with respective amounts of scattering. The one or more machine learning models may be trained to associate imaging settings with respective amounts of scattering in different regions of interest below a surface of a live human tissue. The one or more machine learning models may be trained to detect biological candles and configure the imaging settings based on the detected biological candles. Default imaging settings may be learned for different regions of interest in the body (e.g., associated with different biological candles) to facilitate efficient configuration of imaging parameters. The one or more machine learning models may include a diffusion transformer model architecture, a transformer model architecture, a diffusion model architecture, or other model architecture.

At block 706, the exemplary system input the one or more obtained images into one or more machine learning models. The one or more machine learning models be trained to identify scattering of certain electromagnetic signals comprising each of the one or more obtained images and correct for the scattering to create one or more images of live human tissues at one or more subsurface tissue depths. The one or more machine learning models may be trained to identify micron-scale features of interest within the one or more images of live human tissues at the one or more subsurface tissue depths. The micron-scale feature of interest may include any of a protein accumulation below the surface of the live human tissue, a lipid accumulation below the surface of the live human tissue, and a spectral response of a tissue to the electromagnetic signals. The one or more obtained images may include a plurality of images, and the machine learning model may be trained to correct for the scattering to create a plurality of images of live human tissues at a plurality of subsurface tissue depths.

The one or more machine learning models may be trained by associating training images of known protein accumulations and/or other features of interest (e.g., lipid accumulations, spectral responses, etc.) with subsurface (e.g., subdermal) training images obtained from a sensor positioned at a surface of a live human tissue. The machine learning model may be trained to reconstruct training images of known protein accumulations and/or other features of interest. Training images of known protein accumulations and/or other micron-scale features of interest may include any of fluorescent image(s), magnetic resonance imaging (MRI) image(s), ultrasound image(s), and/or histopathology image(s). The one or more machine learning models used at block 706 may be a module of a machine learning model that includes the machine learning model trained for optimizing imaging settings described above with reference to block 704. The one or more machine learning models used at block 706 may be a separate machine learning model from the machine learning model trained for optimizing imaging settings described above with reference to block 704. The one or more machine learning models used at block 706 may include a transformer architecture, a diffusion architecture, or other model architecture.

At block 708, the exemplary system optionally predicts a disease state. The disease state may include cardiac amyloidosis, carpal tunnel syndrome, atherosclerosis, other forms of amyloidosis (e.g., light chain amyloidosis), peripheral neuropathies (e.g. caused by diabetes), or other disease states identifiable based upon accumulations of proteins, lipids, and/or other features of interest identifiable according to the methods disclosed herein. In some examples, the one or more obtained images may include a time series of images, and the one or more machine learning models may be trained to model and/or predict a progression of a disease.

Process 700 is performed, for example, using one or more electronic devices implementing a software platform. In some embodiments, process 700 is performed using any or all of the aspects of system 100A, system 100B, device 200A, and/or device 200B described above with reference to FIGS. 1A, 1B 2A, and 2B. In some embodiments, process 700 is performed using a client-server system, and the blocks of process 700 are divided up in any manner between the server and one or more client devices. Thus, while portions of process 700 are described herein as being performed by particular devices, it will be appreciated that process 700 is not so limited. In process 700, some blocks are, optionally, combined, the order of some blocks is, optionally, changed, and some blocks are, optionally, omitted. In some examples, additional steps may be performed in combination with the process 700. Accordingly, the operations as illustrated (and described in greater detail above) are exemplary by nature and, as such, should not be viewed as limiting.

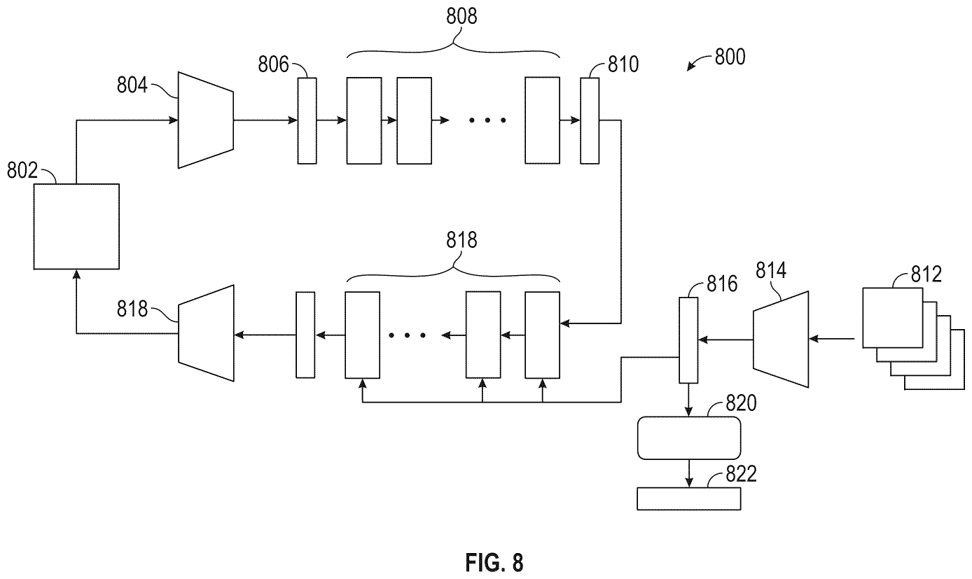

FIG. 8 illustrates an exemplary diagram of a machine learning model 800 that may be implemented in accordance with any of the systems, methods, and devices disclosed herein. Machine learning model 800 may be a latent diffusion model comprising a model architecture similar to that described in Robin Rombach et al., High-Resolution Image Synthesis with Latent Diffusion Models (2022), https://doi.org/10.48550/arXiv.2112.10752, which is incorporated herein by reference in its entirety. Machine learning model 800 may be trained using one or more loss functions. In some examples, machine learning model 800 is trained based on two types of loss functions: reconstructions with sparsity constraints and classification-regressions.

Machine learning model 800 may be a multimodal variant of a latent diffusion model. Machine learning model 800 may include a first encoder 804, a second encoder 814, and/or a decoder 818. Encoder 804 may be trained to produce embeddings 806 (e.g., semantically rich embedding vectors) based on a first input modality 802. The first input modality 802 may include high-resolution ground truth images, such as fluorescent images, 3D complex refractive index maps, MRI images, ultrasound images, and/or other single (or multi) channel 3D volume images. Decoder 818 may be trained to generate corresponding images based on the embeddings 806. The embeddings 806 may be processed using a diffusion model 808. A standard 3D Gaussian process or custom 3D scattering kernel are optionally applied via the diffusion model 808. The custom 3D scattering kernel may be applied to 3D fluorescence and 3D complex refractive index maps The diffusion model 808 may output a noisy embedding 810. Embedding 810 output by the diffusion model 808 may be processed using one or more denoising U-Net blocks 818. The one or more U-Net blocks 818 may be conditioned, using a cross attention mechanism, on one or more embeddings obtained using encoder 814. The one or more embeddings obtained using encoder 814 may be obtained based on a second input modality 812. The second input modality 812 may include images captured under various imaging settings according to aspects of the systems, devices, and methods disclosed herein. For instance, the second input modality 812 may include images captured at different wavelengths of the NIR and/or SWIR spectrum. The second input modality 812 may include images captured using different polarization settings, different illumination settings, or variations of any of the imaging settings disclosed herein. The second input modality 812 may include a plurality of images or image channels. Each or image channel may correspond to a unique configuration of imaging settings. One or more embeddings 816 may be generated for an image or image channel corresponding to each of the unique imaging setting configurations. The cross-attention mechanism may be augmented by a sparsity loss, which may encourage the decoder 818 of machine learning model 800 to minimize the number of image channels (e.g., the number of different imaging setting configurations) needed to obtain accurate image reconstructions. The optimal imaging settings may be determined based on an attention map associated with an output of the decoder 818. In some examples, using cross-attention readout from the model training, the list of illumination parameters that are the most informative are used to reduce the total set of illumination parameters. In some examples, clearing parameters (e.g., for an optical phase clearer) are optimized independently of the illumination parameters once illumination setups are fixed and frozen.

During an inference phase one or more images of the second input modality (e.g., NIR or SWIR images, as described according to any of the various systems, methods, and devices disclosed herein) may be encoded into embeddings 814. The embeddings 814 may be input into decoder 818, and the decoder 818 may generate high-resolution synthetic images of the first modality (e.g., MRI images, ultrasound images, fluorescence images, etc. based on the images of the second modality.

In some examples, one or more of the embeddings 816 obtained using encoder 814 based on the first input modality 812 may be used for one or more classification or regression (e.g., multi-regression) tasks 820. For instance, the embeddings 814 may be processed (e.g., using one or more machine learning models) identify features of interest, including but not limited to protein accumulations, lipid accumulations, inflamed nerves, lymphatic structures, spectral responses of different tissues to the electromagnetic signals, and other structures, microstructures, etc. beneath the surface of a tissue. The embeddings 814 may be processed to predict disease state(s) and/or model disease progressions. The embeddings 814 may be processed to generate additional data modalities.

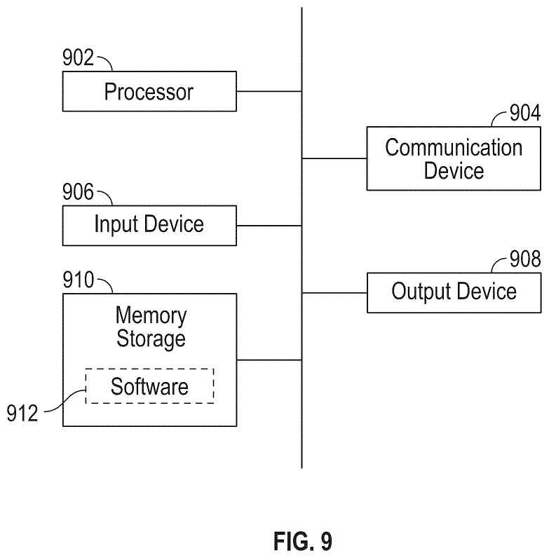

FIG. 9 depicts an exemplary computing device 900, in accordance with one or more examples of the disclosure. Device 900 can be a host computer connected to a network. Device 900 can be a client computer or a server. As shown in FIG. 9, device 900 can be any suitable type of microprocessor-based device, such as a personal computer, workstation, server, or handheld computing device (portable electronic device) such as a phone or tablet. The device can include, for example, one or more of processors 902, input device 906, output device 908, storage 910, and communication device 904. Input device 906 and output device 908 can generally correspond to those described above and can either be connectable or integrated with the computer.

Input device 906 can be any suitable device that provides input, such as a touch screen, keyboard or keypad, mouse, or voice-recognition device. Output device 908 can be any suitable device that provides output, such as a touch screen, haptics device, or speaker.

Storage 910 can be any suitable device that provides storage, such as an electrical, magnetic, or optical memory, including a RAM, cache, hard drive, or removable storage disk. Communication device 904 can include any suitable device capable of transmitting and receiving signals over a network, such as a network interface chip or device. The components of the computer can be connected in any suitable manner, such as via a physical bus or wirelessly.

Software 912, which can be stored in storage 910 and executed by processor 902, can include, for example, the programming that embodies the functionality of the present disclosure (e.g., as embodied in the devices as described above).

Software 912 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 910, that can contain or store programming for use by or in connection with an instruction execution system, apparatus, or device.

Software 912 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 900 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can implement any operating system suitable for operating on the network. Software 912 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

### Example Application(s)

In some examples, the systems, devices, and methods disclosed herein can be used for the characterization of early onset of cardiac amyloidosis (ATTR-cm). ATTR is a severe, progressive, life-threatening systemic disease that causes amyloid fibril deposits in the heart, peripheral nerves and many other organs. The resulting disease has mainly cardiac (ATTR-cm), neurological (ATTR-pn), or both manifestations. ATTR is caused by one of more than 130 aggregate-prone mutations in the transthyretin (TTR) gene, or wild-type TTR protein misfolding and aggregation in older patients. Once considered a rare disease, ATTR is likely severely underdiagnosed.

The majority of ATTR-cm patients develop bi lateral carpal tunnel syndrome 6 years prior to a definitive diagnosis of the disease. This is due to build-up of amyloid fibril deposits in and around the median nerve. MRI, ultrasound imaging, bone scintigraphy and histopathology imaging are the common ways to characterize the disease, as discussed in Martinez-Naharro, A., Baksi, A. J., Hawkins, P. N. et al., Diagnostic imaging of cardiac amyloidosis, NAT REV CARDIOL 17, 413-426 (2020). https://doi.org/10.1038/s41569-020-0334-7, which is incorporated herein by reference in its entirety. Ultrasound imaging of the wrist is the current standard of care to detect swelling of the median nerve, which provides a clue but not a diagnosis of the disease. Bone scintigraphy is another way to detect amyloid deposits in the whole body. Bone scintigraphy requires the injection of a radioactive material to the patient, and is useful to detect amyloid deposits only once the disease has progressed to a point that a patient is very ill.

The systems, devices, and methods disclosed herein can target the carpal tunnel located 1 to 2 cm beneath the skin using NIR and SWIR imaging along with machine-learning based image processing techniques. Whether or not a patient is experiencing carpal tunnel syndrome, the disclosed systems, devices, and methods can detect the presence of amyloid fibril deposits, even in sparse quantities.

### CLAUSES

Clause 1. A method for identifying micron-scale features below a surface of a live human tissue, the method comprising: emitting from one or more emitters at the surface of the live human tissue electromagnetic signals capable of penetrating human tissue of different densities; obtaining from a sensor positioned at the surface of the live human tissue electromagnetic signals reflected from or transmitted through live human tissues of different densities as one or more obtained images; inputting the one or more obtained images into a machine learning model, wherein the machine learning model is trained to: identify scattering of certain electromagnetic signals comprising each of the one or more obtained images; correct for the scattering to create one or more images of live human tissues at one or more subsurface tissue depths; and identify micron-scale features of interest within the one or more images of live human tissues at the one or more subsurface tissue depths.

Clause 2. The method of clause 1, wherein: the emitted electromagnetic signals are comprised of near infrared or short-wave infrared light.

Clause 3. The method of any one of clauses 1-2, comprising modifying a scattering of the electromagnetic signals obtainable by the sensor using a microwave phase array positioned at the surface of the live human tissue.

Clause 4. The method of any one of clauses 1-3, comprising adjusting at least one of an illumination strength and an illumination parameter of the one or more emitters to modify a scattering of the electromagnetic signals obtainable by the sensor.

Clause 5. The method of any one of clauses 1-4, wherein the one or more emitters comprise one or more coherent light sources.

Clause 6. The method of any one of clauses 1-5, wherein the one or more emitters comprise an optical phase array.

Clause 7. The method of any one of clauses 1-6, wherein the sensor comprises a camera configured to capture the near infrared spectrum and the short-wave infrared spectrum.

Clause 8. The method of clause 7, wherein the camera is configured to capture at least part of the visible spectrum.

Clause 9. The method of any one of clauses 1-8, wherein the sensor comprises an optical phase array configured to capture the near infrared spectrum and the short-wave infrared spectrum.

Clause 10. The method of any one of clauses 1-9, wherein the micron scale feature of interest is any of: a protein accumulation below the surface of the live human tissue, a lipid accumulation below the surface of the live human tissue, and a spectral response of a tissue to the electromagnetic signals.

Clause 11. The method of clause 10, wherein the machine learning model has been trained by: associating training images of known protein accumulations with subdermal training images obtained from a sensor positioned at a surface of a live human tissue.

Clause 12. The method of clause 10, wherein the machine learning model has been trained by: training the machine learning model to reconstruct training images of known protein accumulations.

Clause 13. The method of any one of clauses 11-12, wherein the training images of known protein accumulations comprise any one or more of: a fluorescent image; a magnetic resonance imaging (MRI) image; an ultrasound image; and a histopathology image.

Clause 14. The method of any one of clauses 1-13, wherein the machine learning model has been trained to reconstruct from the one or more obtained or created images a three-dimensional model of an anatomy below the surface of the tissue.

Clause 15. The method of any one of clauses 1-14, wherein the machine learning model has been trained to deconvolve a scatter present in the electromagnetic signals received by the one or more image sensors.

Clause 16. The method of any one of clauses 1-15, wherein the one or more image sensors are included on at least one of: a wearable device and an endoscope.

Clause 17. The method of any one of clauses 1-16, comprising: predicting a disease state based on the micron-scale feature of interest.

Clause 18. The method of clause 17, wherein the disease state comprises any of cardiac amyloidosis, carpal tunnel syndrome, light chain amyloidosis, a peripheral neuropathy, or any combination thereof.

Clause 19. The method of any one of clauses 1-18, wherein the protein accumulations below the surface of the tissue are located below the surface of any of: a wrist, an eye, a finger, and a nose.

Clause 20. The method of any one of clauses 1-19, wherein the micron-scale feature of interest is at least one micrometer in size.

Clause 21. The method of any one of clauses 1-20, wherein the surface of the live human tissue is the surface of a skin, a heart, or an eye.

Clause 22. The method of any one of clauses 1-21, wherein the one or more obtained images comprise a plurality of images, and wherein the machine learning model is trained to correct for the scattering to create a plurality of images of live human tissues at a plurality of subsurface tissue depths.

Clause 23. A system for identifying micron-scale features below a surface of a live human tissue, the system comprising: one or more processors, a memory, and one or more programs, wherein the one or more programs are stored in the memory and configured to be executed by the one or more processors, the one or more programs including instructions for: emitting from one or more emitters at the surface of the live human tissue electromagnetic signals capable of penetrating human tissue of different densities; obtaining from a sensor positioned at the surface of the live human tissue electromagnetic signals reflected from or transmitted through live human tissues of different densities as one or more obtained images; inputting the one or more obtained images into a machine learning model, wherein the machine learning model is trained to: identify scattering of certain electromagnetic signals comprising each of the one or more obtained images; correct for the scattering to create one or more images of live human tissues at one or more subsurface tissue depths; and identify micron-scale features of interest within the one or more images of live human tissues at the one or more subsurface tissue depths.

Clause 24. A non-transitory computer-readable storage medium storing one or more programs for identifying micron-scale features below a surface of a live human tissue, the one or more programs comprising instructions, which when executed by one or more processors of an electronic device, cause the electronic device to: emit from one or more emitters at the surface of the live human tissue electromagnetic signals capable of penetrating human tissue of different densities; obtain from a sensor positioned at the surface of the live human tissue electromagnetic signals reflected from or transmitted through live human tissues of different densities as one or more obtained images; input the one or more obtained images into a machine learning model, wherein the machine learning model is trained to: identify scattering of certain electromagnetic signals comprising each of the one or more obtained images; correct for the scattering to create one or more images of live human tissues at one or more subsurface tissue depths; and identify micron-scale features of interest within the one or more images of live human tissues at the one or more subsurface tissue depths.

Clause 25. A device for identifying micron-scale features below a surface of a live human tissue, the device comprising: one or more emitters at the surface of the live human tissue configured to emit electromagnetic signals capable of penetrating human tissue of different densities; at least one sensor positioned at the surface of the live human tissue configured to detect electromagnetic signals reflected from or transmitted through live human tissues of different densities as one or more obtained images; one or more processors; and a memory storing computer instructions that when executed by the one or more processors, cause the device to: input the one or more obtained images into a machine learning model, wherein the machine learning model is trained to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

Clause 26. A method of training a machine learning model to identify micron-scale features from a scan of live human tissues, the method comprising: obtaining one or more images based on electromagnetic signals reflected from or transmitted through live human tissues of different densities; inputting the one or more obtained images into the machine learning model; and training the machine learning model to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

Clause 27. The method of clause 26, wherein the micron scale feature of interest is any of: a protein accumulation, a lipid accumulation, and a spectral response of a tissue to the electromagnetic signals.

Clause 28. The method of clause 27, wherein training the machine learning model comprises: associating training images of known protein accumulations or lipid accumulations with subdermal training images obtained from a sensor positioned at the surface of a live human tissue.

Clause 29. The method of any one of clauses 27-28, wherein training the machine learning model comprises: training the machine learning model to reconstruct training images of known protein accumulations or lipid accumulations.

Clause 30. The method of any one of clauses 28-29, wherein the training images of known protein accumulations or lipid accumulations comprise any one or more of: a fluorescent image; a magnetic resonance imaging (MRI) image; an ultrasound image; and a histopathology image.

Clause 31. The method of any one of clauses 28-30, wherein the training images comprise a training image of a first type depicting a region of a live human tissue and a training image of a second type depicting the same region of the live human tissue.

Clause 32. The method any one of clauses 28-31, wherein at least a subset of the training images are labeled to indicate any of: a disease state, a tissue classification, a biomarker, a segmentation mask of a disease feature, a protein accumulation, a lipid accumulation, and a physiological parameter.

Clause 33. The method of any one of clauses 26-32, wherein training the machine learning model comprises: training the machine learning model to reconstruct from the obtained or created images a three-dimensional model of an anatomy below the surface of the tissue.

Clause 34. The method of any one of clauses 26-33, wherein training the machine learning model comprises: training the machine learning model to deconvolve a scatter present in the electromagnetic signals.

Clause 35. The method of any one of clauses 26-34, wherein obtaining the plurality of images comprises: emitting from one or more emitters at the surface of the live human tissue electromagnetic signals capable of penetrating human tissue of different densities; obtaining from a sensor positioned at the surface of the live human tissue electromagnetic signals reflected from or transmitted through live human tissues of different densities as the one or more obtained images.

Clause 36. The method of clause 35, wherein obtaining the one or more images comprises adjusting at least one of an illumination strength and an illumination parameter of the one or more emitters to modify a scattering of the electromagnetic signals obtainable by the sensor.

Clause 37. The method of clause 36, wherein the illumination parameter comprises any one or more of: an illumination angle, a polarization, a frequency, and a target depth.

Clause 38. The method of any one of clauses 35-37, wherein obtaining the one or more images comprises modifying a scattering of the electromagnetic signals obtainable by the sensor using a microwave phase array positioned at the surface of the live human tissue.

Clause 39. The method of any one of clauses 35-38, wherein the one or more emitters comprise one or more coherent light sources.

Clause 40. The method of any one of clauses 35-39, wherein the one or more emitters comprises an optical phase array.

Clause 41. The method of any one of clauses 35-40, wherein the sensor comprises a camera configured to capture the near infrared spectrum and the short-wave infrared spectrum.

Clause 42. The method of clause 41, wherein the camera is configured to capture at least part of the visible spectrum.

Clause 43. The method of any one of clauses 35-40, wherein the sensor comprises an optical phase array configured to capture the near infrared spectrum and the short-wave infrared spectrum.

Clause 44. The method of any one of clauses 26-43, wherein the electromagnetic signals are comprised of near infrared or short-wave infrared light.

Clause 45. The method of any one of clauses 26-44, wherein the micron scale feature of interest is any of: a protein accumulation below the surface of the live human tissue, a lipid accumulation below the surface of the live human tissue, and a spectral response of a tissue to the electromagnetic signals.

Clause 46. The method of any one of clauses 26-45, wherein the one or more obtained images comprise a plurality of images, and wherein the machine learning model is trained to correct for the scattering to create a plurality of images of live human tissues at a plurality of subsurface tissue depths.

Clause 47. A system for training a machine learning model to identify micron-scale features from a scan of live human tissues, the system comprising: one or more processors, a memory, and one or more programs, wherein the one or more programs are stored in the memory and configured to be executed by the one or more processors, the one or more programs including instructions for: obtaining one or more images based on electromagnetic signals reflected from or transmitted through live human tissues of different densities; inputting the one or more obtained images into the machine learning model; and training the machine learning model to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

Clause 48. A non-transitory computer-readable storage medium storing one or more programs for identifying micron-scale features below a surface of a live human tissue, the one or more programs comprising instructions, which when executed by one or more processors of an electronic device, cause the electronic device to: obtain one or more images based on electromagnetic signals reflected from or transmitted through live human tissues of different densities; input the one or more obtained images into the machine learning model; and train the machine learning model to: identify scattering of certain electromagnetic signals comprising each of the one or more obtained images; correct for such scattering to create one or more images of live human tissues at one or more subsurface tissue depths; and identify micron-scale features of interest within the one or more images of live human tissues at the one or more subsurface tissue depths.

1. A method for identifying micron-scale features below a surface of a live human tissue, the method comprising:
emitting from one or more emitters at the surface of the live human tissue electromagnetic signals capable of penetrating human tissue of different densities;
obtaining from a sensor positioned at the surface of the live human tissue electromagnetic signals reflected from or transmitted through live human tissues of different densities as one or more obtained images;
inputting the one or more obtained images into a machine learning model, wherein the machine learning model is trained to:
identify scattering of certain electromagnetic signals comprising each of the one or more obtained images;
correct for the scattering to create one or more images of live human tissues at one or more subsurface tissue depths; and
identify micron-scale features of interest within the one or more images of live human tissues at the one or more subsurface tissue depths.

2. The method of claim 1, wherein:
the emitted electromagnetic signals are comprised of near infrared or short-wave infrared light.

3. The method of claim 1, comprising modifying a scattering of the electromagnetic signals obtainable by the sensor using a microwave phase array positioned at the surface of the live human tissue.

4. The method of claim 1, comprising adjusting at least one of an illumination strength and an illumination parameter of the one or more emitters to modify a scattering of the electromagnetic signals obtainable by the sensor.

5. The method of claim 1, wherein the one or more emitters comprise one or more coherent light sources.

6. The method of claim 1, wherein the one or more emitters comprise an optical phase array.

7. The method of claim 1, wherein the sensor comprises a camera configured to capture the near infrared spectrum and the short-wave infrared spectrum.

8. The method of claim 7, wherein the camera is configured to capture at least part of the visible spectrum.

9. The method of claim 1, wherein the sensor comprises an optical phase array configured to capture the near infrared spectrum and the short-wave infrared spectrum.

10. The method of claim 1, wherein the micron scale feature of interest is any of: a protein accumulation below the surface of the live human tissue, a lipid accumulation below the surface of the live human tissue, and a spectral response of a tissue to the electromagnetic signals.

11. The method of claim 10, wherein the machine learning model has been trained by:
associating training images of known protein accumulations with subdermal training images obtained from a sensor positioned at a surface of a live human tissue.

12. The method of claim 10, wherein the machine learning model has been trained by:
training the machine learning model to reconstruct training images of known protein accumulations.

13. The method of claim 11, wherein the training images of known protein accumulations comprise any one or more of:
a fluorescent image;
a magnetic resonance imaging (MRI) image;
an ultrasound image; and
a histopathology image.

14. The method of claim 1, wherein the machine learning model has been trained to reconstruct from the one or more obtained or created images a three-dimensional model of an anatomy below the surface of the tissue.

15. The method of claim 1, wherein the machine learning model has been trained to deconvolve a scatter present in the electromagnetic signals received by the one or more image sensors.

16. The method of claim 1, wherein the one or more image sensors are included on at least one of: a wearable device and an endoscope.

17. The method of claim 1, comprising: predicting a disease state based on the micron-scale feature of interest.

18. The method of claim 17, wherein the disease state comprises any of cardiac amyloidosis, carpal tunnel syndrome, light chain amyloidosis, a peripheral neuropathy, or any combination thereof.

19. The method of claim 1, wherein the protein accumulations below the surface of the tissue are located below the surface of any of: a wrist, an eye, a finger, and a nose.

20. The method of claim 1, wherein the micron-scale feature of interest is at least one micrometer in size.

21. The method of claim 1, wherein the surface of the live human tissue is the surface of a skin, a heart, or an eye.

22. The method of claim 1, wherein the one or more obtained images comprise a plurality of images, and wherein the machine learning model is trained to correct for the scattering to create a plurality of images of live human tissues at a plurality of subsurface tissue depths.

23. A system for identifying micron-scale features below a surface of a live human tissue, the system comprising: one or more processors, a memory, and one or more programs, wherein the one or more programs are stored in the memory and configured to be executed by the one or more processors, the one or more programs including instructions for:
emitting from one or more emitters at the surface of the live human tissue electromagnetic signals capable of penetrating human tissue of different densities;
obtaining from a sensor positioned at the surface of the live human tissue electromagnetic signals reflected from or transmitted through live human tissues of different densities as one or more obtained images;
inputting the one or more obtained images into a machine learning model, wherein the machine learning model is trained to:
identify scattering of certain electromagnetic signals comprising each of the one or more obtained images;
correct for the scattering to create one or more images of live human tissues at one or more subsurface tissue depths; and
identify micron-scale features of interest within the one or more images of live human tissues at the one or more subsurface tissue depths.

24. A non-transitory computer-readable storage medium storing one or more programs for identifying micron-scale features below a surface of a live human tissue, the one or more programs comprising instructions, which when executed by one or more processors of an electronic device, cause the electronic device to:
emit from one or more emitters at the surface of the live human tissue electromagnetic signals capable of penetrating human tissue of different densities;
obtain from a sensor positioned at the surface of the live human tissue electromagnetic signals reflected from or transmitted through live human tissues of different densities as one or more obtained images;
input the one or more obtained images into a machine learning model, wherein the machine learning model is trained to:
identify scattering of certain electromagnetic signals comprising each of the one or more obtained images;
correct for the scattering to create one or more images of live human tissues at one or more subsurface tissue depths; and
identify micron-scale features of interest within the one or more images of live human tissues at the one or more subsurface tissue depths.

25. A device for identifying micron-scale features below a surface of a live human tissue, the device comprising:
one or more emitters at the surface of the live human tissue configured to emit electromagnetic signals capable of penetrating human tissue of different densities;
at least one sensor positioned at the surface of the live human tissue configured to detect electromagnetic signals reflected from or transmitted through live human tissues of different densities as one or more obtained images;
one or more processors; and
a memory storing computer instructions that when executed by the one or more processors, cause the device to:
input the one or more obtained images into a machine learning model, wherein the machine learning model is trained to:
identify scattering of certain electromagnetic signals comprising each of the one or more obtained images;
correct for such scattering to create one or more images of live human tissues at one or more subsurface tissue depths; and
identify micron-scale features of interest within the one or more images of live human tissues at the one or more subsurface tissue depths.
